(19) United States
(12) Patent Application Publication
(10) Pub. No.: US 2026/0106025 A1
(43) Pub. Date: Apr. 16, 2026

(54) Computer Vision Based Assessment of Infant Face and Body Symmetry

(71) Applicant: Northeastern University, Boston, MA (US)

(72) Inventors: Sarah OSTADABBAS, Watertown, MA (US); Michael WAN, Brunswick, ME (US); Xiaofei HUANG, Lynnfield, MA (US); Lingfei LUAN, Bowling Green, OH (US); Bethany TUNIK, Windham, NH (US)

(21) Appl. No.: 19/112,461

(22) PCT Filed: Nov. 13, 2023
(86) PCT No.: PCT/US2023/037207

§ 371 Date: Mar. 17, 2025

Related U.S. Application Data
(60) Provisional application No. 63/424,431, filed on Nov. 10, 2022.

(51) Int. Cl. G16H 50/20; G06T 7/68; G06V 40/16

(52) U.S. Cl. CPC G16H 50/20; G06T 7/68; G06V 40/168

(57) ABSTRACT

Provided herein are methods and systems for screening, diagnosing, or monitoring a disease or condition in a human infant including providing one or more images of the infant, selecting at least two geometric symmetry measures of the infant indicative of the disease or condition, analyzing, using both an infant face landmark estimation model and an infant body landmark estimation model of a computer vision system, the one or more images to produce a plurality of landmark values corresponding to the geometric symmetry measures, determining, by the computer vision system, a geometry of one or more facial structures of the infant and/or a 3D body pose of the infant based on the plurality of landmark values, and quantifying, based on the geometry of the one or more facial structures of the infant and/or the 3D body pose of the infant, a symmetry of the geometric symmetry measures.

### CROSS REFERENCE TO RELATED APPLICATIONS

This application claims benefit under 35 U.S.C. § 119(e) of U.S. Provisional Application No. 63/424,431, filed on 10 Nov. 2022, entitled “Computer Vision Based Assessment of Infant Face and Body Symmetry.” the entirety of which is incorporated by reference herein.

### STATEMENT REGARDING FEDERALLY SPONSORED RESEARCH OR DEVELOPMENT

This invention was made with government support under Grant Number 2143882 awarded by the National Science Foundation. The government has certain rights in the invention.

### BACKGROUND

Bilateral postural symmetry plays a key role as a potential risk marker for autism spectrum disorder (ASD) and as a symptom of congenital muscular torticollis (CMT) in infants, but current methods of assessing symmetry for screening, diagnosis, and monitoring for CMT or ASD require laborious professional assessments. Due to the labor-intensiveness and cost associated with such professional assessments, they cannot be performed in a timely manner in all instances. Thus, critical time is often lost in diagnosing and monitoring CMT, resulting in delayed interventions and, consequently, degraded patient outcomes.

These challenges are not insignificant. Torticollis is a common condition in infants and children, characterized by a persistent neck tilt or twist to one side. Its most common form, congenital muscular torticollis (CMT), has an estimated incidence of 3.9% to 16%. Early treatment is critical: outcomes are best when CMT is diagnosed and physical therapy treatment started before the infant is three months old, and conversely, if untreated or treated later, CMT can lead to face, skull, or spine deformities, pain and limited motion, and the need for invasive interventions and surgery.

Recently, computer vision technology has been developed for studying infant face and body poses for health and developmental applications. Such computer vision has begun to be used in the field of automated medical diagnosis, including to distinguish atypical development through video-based behavior monitoring.

For example, as part of the behavioral phenotyping for autism spectrum disorder (ASD), the inventors previously examined the arm movement and asymmetry in children. They extracted arm and shoulder angles of the child from recorded videos, using a pre-trained real-time multi-person 2D pose estimation model, OpenPose. A computer vision tool to measure and identify ASD behavioral markers based on components of the autism observation has been previously introduced. The inventors first applied 2D pose estimation, which is proposed by extending the Object Cloud Model (OCM) segmentation framework to work with video data, and to produce a 2D stick-man of the toddlers in video segments in which they were walking naturally. Then static and dynamic arm symmetry, as one type of the behavior marker, was detected using the absolute 2D angle difference between corresponding arm parts across time in video segments. Asymmetry was defined if the angle between two corresponding arm parts differs by more than 45°. This prior work detected body movement symmetry based on the measured angle differences of arm pairs.

Meanwhile, some of the inventors have separately previously developed a virtual reality (VR)-based motor intervention methodology by using motion tracking data to quantify efficiency, synchrony and symmetry of whole-body movement. In this manner the inventors proposed another kind of hand bilateral symmetry definition, which is the average and standard deviation of the difference in absolute value of horizontal distance between the hands. For symmetry measurement, the 2D locations of wrists were predicted by the pose estimator integrated in the Microsoft Kinect API and then the symmetry score was calculated according to their proposed symmetry measurement formula.

However, a universal shortcoming of all previous computer vision-based approaches to postural symmetry is their reliance on measurements from 2D body poses, even though human body movement and symmetry is fundamentally three-dimensional. Postural symmetry measurement via 3D body poses has yet to be explored.

### SUMMARY

Described herein are systems and methods for computer vision-based assessment of infant face and body symmetry. In some embodiments, a computer vision based infant symmetry assessment system can be provided which leverages 3D human pose estimation for infants (also referred to herein as “3D pose-based symmetry assessment system”). Furthermore, as the inventors discovered during development of the described computer vision based infant symmetry assessment system, evaluation and calibration of the system against ground truth assessments is complicated by the inventors' findings, from an analysis of human ratings of angle and symmetry, that such human ratings exhibit low inter-rater reliability. Therefore, to rectify this shortcoming, also described herein is a newly developed Bayesian estimator of the ground truth derived from a probabilistic graphical model of fallible human raters.

It is shown herein that the described 3D infant pose estimation model described herein can achieve 68% area under the receiver operating characteristic curve performance in predicting the Bayesian aggregate labels, compared to only 61% from a 2D infant pose estimation model and 60% from a 3D adult pose estimation model, highlighting the importance of 3D poses and infant domain knowledge in assessing infant body symmetry. This analysis also suggests that human ratings are susceptible to higher levels of bias and inconsistency, and hence the 3D pose-based symmetry assessment system is calibrated, but not directly supervised, by Bayesian aggregate human ratings, yielding higher levels of consistency and lower levels of inter-limb assessment bias.

In addition, as a first step towards applying such a 3D pose-based symmetry assessment system, also described herein is an analysis of the viability of using such computer vision techniques to assess a set of geometric measures of symmetry in the face and upper body, previously identified in the medical literature as being relevant to CMT or similarly presenting ocular torticollis condition (non-congenital), purely from casual photographs captured from infants in their natural environments.

In one aspect, a method of screening, diagnosing, or monitoring a disease or condition in a human infant is provided. The method includes providing one or more images of the infant. The method also includes selecting at least two geometric symmetry measures of the infant indicative of the disease or condition. The method also includes analyzing, using both an infant face landmark estimation model and an infant body landmark estimation model of a computer vision system, the one or more images to produce a plurality of landmark values corresponding to the geometric symmetry measures. The method also includes determining, by the computer vision system, a geometry of one or more facial structures of the infant and/or a 3D body pose of the infant based on the plurality of landmark values. The method also includes quantifying, based on the geometry of the one or more facial structures of the infant and/or the 3D body pose of the infant, a symmetry of the geometric symmetry measures.

In some embodiments, the disease or condition is torticollis, such as congenital muscular torticollis (CMT), autism spectrum disorder (ASD), or cerebral palsy (CP). In some embodiments, the step of quantifying includes determining bilateral postural asymmetry. In some embodiments, the step of quantifying also includes using a symmetry classifier to assess, based on body joint angles obtained from the 3D pose determination, one or more symmetry ratings corresponding to the geometric symmetry measures. In some embodiments, the method also includes aggregating, using a Bayesian estimator, a plurality of annotated symmetry ratings corresponding to the one or more symmetry ratings to establish one or more aggregated Bayesian ground truths. In some embodiments, the method also includes comparing each of the symmetry ratings to a corresponding one of the aggregated Bayesian ground truths to calibrate the computer vision system. In some embodiments, the method also includes determining, by a maximum-a-posteriori (MAP) estimator, a performance standard applicable to the annotated symmetry ratings. In some embodiments, the method also includes evaluating, by applying an expectation maximization algorithm, a performance relative to the performance standard of a rater associated with each of the annotated symmetry ratings. In some embodiments, the step of aggregating, by the Bayesian estimator, also includes weighting each of the annotated symmetry ratings according to the evaluated rater performance. In some embodiments, the evaluated performance is at least partially determined according to an average Cohen's κ agreement between each rater and the other raters.

In some embodiments, the geometric symmetry measures include one or more of orbit slopes angle, relative face size, face angle, gaze angle, translational deformity, habitual head deviation, or combinations thereof. In some embodiments, the step of analyzing also includes producing, by the infant face landmark estimation model, produce 68 landmark values. In some embodiments, the step of analyzing also includes producing, by the infant body landmark estimation model, a plurality of landmark values corresponding to one or more of an upper arm, a lower arm, an upper leg, a lower leg, or combinations thereof of the infant. In some embodiments, the plurality of landmark values produced by the infant body landmark estimation model correspond to one or more pairs of limbs of the infant, the one or more pairs of limbs of the infant including one or more of an upper right arm and upper left arm pair, a lower right arm and lower left arm pair, an upper right leg and upper left leg pair, a lower right leg and lower left leg pair, or combinations thereof. In some embodiments, the step of quantifying a symmetry of the of the geometric symmetry measures also includes determining an angle difference across each of the one or more pairs of limbs. In some embodiments, the method also includes comparing the angle difference for each of the one or more pairs of limbs to a corresponding threshold angle. In some embodiments, the method also includes classifying each of the pairs of limbs as symmetrical or asymmetrical. In some embodiments, a symmetrical classification indicates that a corresponding one of the pairs of limbs has a determined angle difference within the corresponding angle threshold. In some embodiments, an asymmetrical classification indicates that the corresponding one of the pairs of limbs has a determined angle difference exceeding the corresponding angle threshold. In some embodiments, the method also includes assigning each of the pairs of limbs to an angle class. In some embodiments, the angle classes include one or more of <30°, ≥30°, <60°, ≥60°, 30°-59°, or combinations thereof.

Additional features and aspects of the technology include the following:

1. A method of screening, diagnosing, or monitoring a disease or condition in a human infant, the method comprising providing one or more images of the infant;
selecting at least two geometric symmetry measures of the infant indicative of the disease or condition;
analyzing, using both an infant face landmark estimation model and an infant body landmark estimation model of a computer vision system, the one or more images to produce a plurality of landmark values corresponding to the geometric symmetry measures;
determining, by the computer vision system, a geometry of one or more facial structures of the infant and/or a 3D body pose of the infant based on the plurality of landmark values; and
quantifying, based on the geometry of the one or more facial structures of the infant and/or the 3D body pose of the infant, a symmetry of the geometric symmetry measures.

2. The method of feature 1, wherein the disease or condition is torticollis, such as congenital muscular torticollis (CMT), autism spectrum disorder (ASD), or cerebral palsy (CP).

3. The method of any of features 1-2, wherein the step of quantifying comprises determining bilateral postural asymmetry.

4. The method of any of features 1-3, wherein the step of quantifying further comprises using a symmetry classifier to assess, based on body joint angles obtained from the 3D pose determination, one or more symmetry ratings corresponding to the geometric symmetry measures.

5. The method of feature 4, further comprising aggregating, using a Bayesian estimator, a plurality of annotated symmetry ratings corresponding to the one or more symmetry ratings to establish one or more aggregated Bayesian ground truths.

6. The method of feature 5, further comprising comparing each of the symmetry ratings to a corresponding one of the aggregated Bayesian ground truths to calibrate the computer vision system.

7. The method of any of features 5-6, further comprising:
determining, by a maximum-a-posteriori (MAP) estimator, a performance standard applicable to the annotated symmetry ratings; and
evaluating, by applying an expectation maximization algorithm, a performance relative to the performance standard of a rater associated with each of the annotated symmetry ratings.

8. The method of feature 7, wherein the step of aggregating, by the Bayesian estimator, further comprises weighting each of the annotated symmetry ratings according to the evaluated rater performance.

9. The method of any of features 7-8, wherein the evaluated performance is at least partially determined according to an average Cohen's κ agreement between each rater and the other raters.

10. The method of any of features 1-9, wherein the geometric symmetry measures include one or more of orbit slopes angle, relative face size, face angle, gaze angle, translational deformity, habitual head deviation, or combinations thereof.

11. The method of any of features 1-10, wherein the step of analyzing further comprises producing, by the infant face landmark estimation model, produce 68 landmark values.

12. The method of any of features 1-11, wherein the step of analyzing further comprises producing, by the infant body landmark estimation model, a plurality of landmark values corresponding to one or more of an upper arm, a lower arm, an upper leg, a lower leg, or combinations thereof of the infant.

13. The method of feature 12, wherein the plurality of landmark values produced by the infant body landmark estimation model correspond to one or more pairs of limbs of the infant, the one or more pairs of limbs of the infant including one or more of an upper right arm and upper left arm pair, a lower right arm and lower left arm pair, an upper right leg and upper left leg pair, a lower right leg and lower left leg pair, or combinations thereof.

14. The method of feature 13, wherein the step of quantifying a symmetry of the of the geometric symmetry measures further comprises determining an angle difference across each of the one or more pairs of limbs.

15. The method of feature 14, further comprising comparing the angle difference for each of the one or more pairs of limbs to a corresponding threshold angle.

16. The method of feature 15, further comprising classifying each of the pairs of limbs as symmetrical or asymmetrical, wherein;
a symmetrical classification indicates that a corresponding one of the pairs of limbs has a determined angle difference within the corresponding angle threshold; and
an asymmetrical classification indicates that the corresponding one of the pairs of limbs has a determined angle difference exceeding the corresponding angle threshold.

17. The method of any of features 14-16, further comprising assigning each of the pairs of limbs to an angle class.

18. The method of feature 17, wherein the angle classes include one or more of <30°, ≥30°, <60°, ≥60°, 30°-59°, or combinations thereof.

### BRIEF DESCRIPTION OF THE DRAWINGS

FIGS. 1A-1D and 1E-1H illustrate two examples of discrepancies between 2D pose-based and 3D pose-based symmetry measurements. In each example, four limb pairs are annotated in different colors based on the corresponding symmetry label. If the limb pair is symmetric, both sides of the limb parts are marked in green, otherwise, they are in red. Parts of the body skeleton not comprising the four limb pairs are uniformly plotted in gray. In particular, FIGS. 1A and 1E illustrate a Bayesian aggregated symmetry result from human ratings as weak ground truth on the raw image (occluded limb parts are not shown). FIGS. 1B and 1F illustrate 2D pose-based measurement results on a 2D predicted skeleton. FIGS. 1C, 1D, 1G, and 1H illustrate the 3D pose-based measurement results on a 3D predicted skeleton at different viewing angles.

FIG. 2 illustrates an infant pose symmetry measurement. A right upper arm RA_{U} and lower arm RA_{L} are mirrored across a mid-perpendicular line Pₛ (in 2D) or plane (in 3D) (shown in larger dashed lines) of a line segment lₛ connecting two shoulder joints. RA_{U} and RA_{L} are aligned with their left counterparts, LA_{U} and LA_{L}, at the root joints, resulting in the reference limbs REF (shown in small, dashed lines). The right upper and lower legs are likewise mirrored across a mid-perpendicular Pₕ of a line segment lₕ connecting two hip joints and aligned with their left counterparts. All four resulting angles θ are measured, and, for each angle, the limb pair is considered pose symmetric if the calculated angle is less than a predefined threshold.

FIGS. 3A-3B illustrate average Cohen's κ agreement of a given individual assessment with 10 human rater assessments (with self-agreement excluded for the human raters) for both Angle Class (FIG. 3A) and Symmetry (FIG. 3B) assessments. Among human raters, Raters 5 and 10 stand out as outliers for angle assessment, as does Rater 5 for symmetry assessments. The Bayesian aggregate assessment exhibits high average agreement, as expected, but the human-voted assessment does not. Among the pose-based assessments, those derived from 3D ground truth or 3D predicted poses by using HW-HuP model agree most strongly with human assessments, especially for the more objective assessment of angle level. Since the angle class is ordered, the quadratically weighted Cohen's κ is used for those assessments.

FIG. 4 illustrates Cohen's κ agreements between limbs of human, Bayesian aggregate, and 3D pose estimation, and 3D weak ground truth based assessments for angle and symmetry. While some level of inter-limb agreement may exist in the actual (inaccessible) ground truth data, the high agreements in human assessment of symmetry seem particularly excessive and are likely attributable to bias.

FIG. 5 illustrates Spearman's ρ ranked correlation between each assessor's angle and asymmetry assessments across all infants and limb pairs. Assessments with high scores can be interpreted as enjoying high “internal consistency.” Low scores can be caused either by low internal consistency or by angle threshold misalignment (as with the 3D adult pose-based model).

FIGS. 6A-6B illustrate receiver operating characteristic (ROC) curves for logistic regression of the Bayesian aggregate angle class (FIG. 6A) and symmetry (FIG. 6B), from raw angles from the listed predicted or ground truth sources, across 2800 pairs of limbs in the infant image set. The regression based on the weak 3D ground truth yields best results, while the 3D infant pose estimation model performs better than the other estimation models or the 2D ground truth. The angle classes have been combined into two groups [<30, 30], for simplicity. Corresponding areas under the curve (AUCs) can be found in Table 2.

FIGS. 7A-7F illustrate Cohen's κ agreement between various pose estimation-based assessments of symmetry and human assessments, as the threshold angle for defining the threshold for pose base assessment varies. Agreement with human assessment is either given as the mean of agreement with each of the 10 raters, or as agreement with the voted or Bayesian aggregate rater. These results show that the highest capacity for agreement is afforded by the Bayesian aggregate rater on the one hand, and the 3D infant pose estimation-based model on the other.

FIGS. 8A-8D illustrate an example demonstrating the effect of angle threshold on pose-based measurement results and constraints on the feasibility of 2D pose-based method. Using the same labeling system as in FIGS. 1A-1H, four limb pairs are annotated in different colors based on the corresponding symmetry label. If the limb pair is symmetric, both sides of the limb parts are marked in green, otherwise, they are in red. Parts of the body skeleton not comprising the four limb pairs are uniformly plotted in gray. In particular, FIG. 8A illustrates a Bayesian aggregated symmetry result from human ratings as weak ground truth on the raw image, FIG. 8B illustrates 2D pose-based assessment results on a 2D predicted skeleton, and FIG. 8C illustrates the 3D pose-based measurement results on a 3D predicted skeleton at different viewing angles.

FIGS. 9A-9B illustrate mean assessments of symmetry level by assessed angle difference level, for all 10 raters plus the Bayesian aggregate rater and the predicted 2D-and 3D-pose-based models. Means are taken over all 700 SyRIP infant images per each of four pairs of limbs, with confidence intervals of one standard deviation at each angle level indicated. These statistics reveal wide variance in determination of symmetry versus angle difference across human raters, although most raters are fairly consistent across limbs. An upwardly sloped segment, as seen most prominently in Rater 8's upper arm assessments and or Rater 10's lower arm assessments, indicates an apparent inconsistency in aggregate assessments. As shown, while small confidence intervals indicate consistent assessments, the converse does not necessarily hold.

FIG. 10A-10D illustrate distribution of raw angle differences, across four pairs of limbs (upper arm, lower arm, upper leg, and lower leg) and 700 real SyRIP infant images, as reported by a range of pose-based models. Models based on 3D poses yield far more consistent and seemingly realistic angles, compared with models based on 2D poses.

FIGS. 11A-11B illustrate a comparison of the performance of pose-based symmetry measurement results, wherein symmetry assessments for each pair of limbs are compared between SyRIP 2D ground truth pose (2D GT) and weak 3D ground truth pose (3D GT).

FIGS. 11C-11D illustrate a comparison of the performance of pose-based symmetry measurement results, wherein symmetry assessments for each pair of limbs are compared between 3D predicted pose using infant HW-HuP model (3D Infant Est.) vs. weak 3D ground truth pose (3D GT).

FIGS. 11E-11F illustrate a comparison of the performance of pose-based symmetry measurement results, wherein symmetry assessments for each pair of limbs are compared between 3D predicted pose by using adult SPIN model (3D Adult Est.) vs. 3D predicted pose by using infant HW-HuP model (3D Infant Est.). For each of FIGS. 11A-11F, Bayesian aggregate results are overlaid on the original images, as a weak ground truth and the labeling system is the same as in FIGS. 1A-1H. As shown across FIGS. 11A-11F, 3D infant pose estimation yields better results than those obtained from the 3D adult pose estimation, but the best results come from the weak 3D ground truth. The Bayesian result of left-side example in FIG. 11E is incorrect, possibly, due to the effects of occlusion on human judgement. The pose-based methods described herein have been trained to be robust to occlusion, and can produce objective evaluations where human assessments falter.

FIG. 12 illustrates a schematic of a range of geometric facial and upper body measures of symmetry, which are drawn from medical research literature on torticollis in infants and children, along with a schematic of facial and upper body landmark estimates generated by deep learning computer vision techniques in the infant domain. As described herein, experimental evaluation shows that application of these deep learning computer vision techniques to such infant and child torticollis measures yields better results than landmark estimation methods largely trained on adult data.

FIG. 13 illustrates face and body (shoulder) landmarks available from ground truth annotations, with index numbers for each landmark point in correspondence with the definitions in Table 7 (shown in FIG. 17).

FIGS. 14A-14B illustrate scatter plots of predictions vs. ground truth for six geometric measures of symmetry (definitions given in Table 7, shown in FIG. 17), with the scale chosen to emphasize the strong effect of outliers on the predictions derived from adult pose estimation models in FIG. 14B compared to those derived from the infant pose estimation models in FIG. 14A. Table 9 provides a more quantitative characterization of performance for each quantity. Lines are drawn for reference.

TABLE 9
ESTIMATION PERFORMANCE OF GEOMETRIC MEASURES OF
SYMMETRY FROM INFANT AND ADULT-BASED MODELS

| Metric | | Models | osa | rfs | fa | ga | td | hhd |
|---|---|---|---|---|---|---|---|---|
| Spearman's ρ | ↑ | Infant | 0.36 * | 0.61 *** | 0.60 *** | 0.79 *** | 0.53 *** | 0.80 *** |
| | | Adult | −0.10 | 0.58 *** | 0.53 *** | 0.78 *** | 0.36 * | 0.78 *** |
| BCA (%) | ↑ | Infant | 50.0 | 77.8 | 75.0 | 80.6 | 88.9 | 77.8 |
| | | Adult | 41.7 | 75.0 | 66.7 | 77.8 | 69.4 | 77.8 |
| MAE | ↓ | Infant | 1.98 | 0.04 | 2.18 | 2.29 | 0.06 | 2.63 |
| | | Adult | 4.73 | 0.07 | 4.51 | 5.25 | 0.08 | 4.71 |
| RMSE | ↓ | Infant | 2.70 | 0.05 | 3.38 | 3.28 | 0.09 | 3.51 |
| | | Adult | 12.88 | 0.17 | 11.27 | 15.61 | 0.12 | 11.71 |

| | | | | | | |
|---|---|---|---|---|---|---|
| Measure type | ∘ | ratio | ∘ | ∘ | ratio | ∘ |
| Ground truth mean | 0.80 | 1.00 | −0.34 | 87.64 | 0.11 | −2.62 |

* p ≤ 0.05,
** p ≤ 0.01,
*** p ≤ 0.001;
best performance in bold

FIGS. 15A-15B illustrate ground truth, infant model predicted positions, and adult model predicted positions of various geometric elements related to measures of symmetry, superimposed on the underlying face and shoulder landmark estimations. The geometric elements were chosen to illustrate outlier cases of bad facial landmark predictions, which disproportionally contribute to the poor quantitative performance from the pose estimation-based models, especially for the models trained on adult data. Outside of these outlier cases, performance is difficult to adjudicate by visual inspection, and thus results analysis discussed herein is guided by quantitative performance metrics.

FIG. 16 illustrates Table 1, a list of data types associated with each infant image described herein. The underlying 700 real infant images are sourced from the synthetic and real infant pose (SyRIP) dataset.

FIG. 17 illustrates Table 7, defining geometric measures of face and upper body symmetry pertaining to torticollis.

### DETAILED DESCRIPTION

As noted above, bilateral postural symmetry plays a key role as a potential risk marker for autism spectrum disorder (ASD) and as a symptom of congenital muscular torticollis (CMT) in infants. Symmetry assessment can also be important in the screening, diagnosis, and monitoring of other common neurodevelopmental conditions, including, for example, cerebral palsy (CP). However, current methods of assessing symmetry require laborious clinical expert assessments, which, as described below, can be unreliable and inconsistent.

Provided herein are methods and systems for computer vision-based assessment of infant face and body symmetry. In some embodiments, a computer vision based infant symmetry assessment system can be provided which leverages 3D human pose estimation for infants (also referred to herein as “3D pose-based symmetry assessment system”). Furthermore, as the inventors discovered during development of the described computer vision based infant symmetry assessment system, evaluation and calibration of the system against ground truth assessments is complicated by the inventors' findings, from an analysis of human ratings of angle and symmetry, that such human ratings exhibit low inter-rater reliability. Therefore, to rectify this shortcoming, also described herein is a newly developed Bayesian estimator of the ground truth derived from a probabilistic graphical model of fallible human raters.

It is shown herein that the described 3D infant pose estimation model described herein can achieve 68% area under the receiver operating characteristic curve performance in predicting the Bayesian aggregate labels, compared to only 61% from a 2D infant pose estimation model and 60% from a 3D adult pose estimation model, highlighting the importance of 3D poses and infant domain knowledge in assessing infant body symmetry. This analysis also suggests that human ratings are susceptible to higher levels of bias and inconsistency, and hence the 3D pose-based symmetry assessment system is calibrated, but not directly supervised, by Bayesian aggregate human ratings, yielding higher levels of consistency and lower levels of inter-limb assessment bias.

In addition, as a first step towards applying such a 3D pose-based symmetry assessment system, also described herein is an analysis of the viability of using such computer vision techniques to assess a set of geometric measures of symmetry in the face and upper body, previously identified in the medical literature as being relevant to CMT or similarly presenting ocular torticollis condition (non-congenital), purely from casual photographs captured from infants in their natural environments.

### Incongruent Annotations and Computer Vision Based Symmetry Assessment

The computer vision based infant symmetry assessment system described herein can be used for assessing bilateral infant postural symmetry from images, based on 3D human pose estimation, domain adapted to the challenging setting of infant bodies. Analysis indicates that the system is less susceptible to inter-limb biases present in human ratings, and as such could be used to great effect in telehealth, where even experts might find it difficult to judge 3D symmetry from on-screen 2D images. Since the system is based on angles extracted from pose estimation, it is both privacy-preserving and highly interpretable, and is adaptable to new definitions of postural symmetry based on updated scientific hypotheses or discoveries, as well as for different conditions.

The system assesses bilateral postural asymmetry by first using state-of-the-art 3D body pose estimation designed specifically for infant bodies and secondly learning a pose-based assessment calibrated to human ratings of asymmetry. The pipeline is simple but its implementation is highly nontrivial because reliable ground truth data does not exist for either task. In particular, for pose estimation, there are no infant datasets labeled with 3D ground truth poses, which would require an apparatus that is infeasible for use with infant subjects. Instead, such pose estimation can be achieved by expanding an existing infant body dataset with new 3D pose labels obtained by manual correction of predictions attained from a 3D infant pose estimation model. Nonetheless, as this 3D data is guided only by perception from flat images, these labels can only serve as weak 3D ground truth.

As for symmetry assessment, a survey of 10 human raters was made with respect to their assessments of pose symmetry and angle differences in four pairs of limbs across 700 infant images. Analysis of that survey revealed low inter-rater reliability as well as suggestions of low internal consistency and high bias. Thus, in both settings, ground truth data is constrained by the fundamental challenge of deriving three-dimensional information from two-dimensional images, especially in the domain of infant bodies.

The overall approach in order to overcoming these challenges was to effectively “bootstrap” from kernels of reliable information in both tasks to obtain globally reliable and bias-free computer vision assessments of body symmetry. Specifically, the strategy included the following elements.

First, to remedy the unreliability of the human raters, a probabilistic graphical model of the human raters as fallible assessors was employed, and a Bayesian aggregate of the underlying ground truth was computed, which exhibits a higher level of internal consistency than the human ratings it is derived from, although full reliability is not assumed.

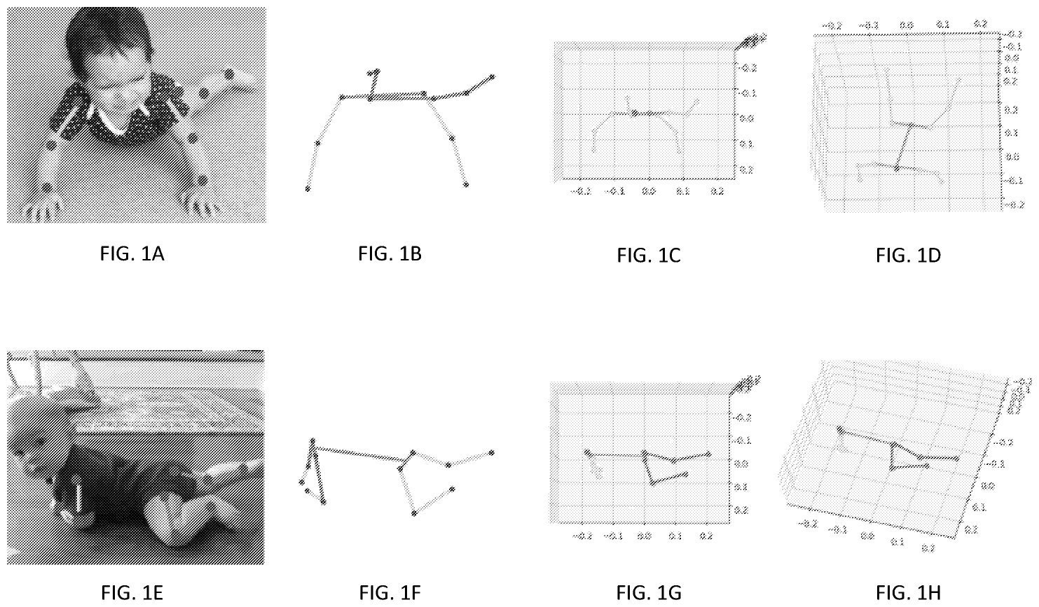

Second, for infant images, the body joint angles obtained from the infant 3D pose estimation model can be used alone to predict the Bayesian ground truth assessments on those images with reasonable accuracy, about 68% area under the receiver operating characteristic (ROC) curve. By comparison, use of angles obtained from an infant 2D pose estimation model only achieves 61% area under the ROC curve. Some visualized examples of this discrepancy are shown in FIG. 1. The power to predict a response variable obtained completely independently, and despite potential noise in both variables, provides evidence of the accuracy of the 3D pose estimated angles.

Third, a simple symmetry classifier for infant images is created based on the 3D pose estimated angles, now known to be fairly accurate, as calibrated by the Bayesian aggregate symmetry rating. This classifier is guided by human intuitions of cutoff thresholds for symmetry assessment but, by design and as quantitatively verified, is free from the apparent biases stemming from errant factors which affect human judgement. The classifier's superiority over an analogous classifier derived from 2D pose estimates is also demonstrated.

Taken together, despite the challenges of both human assessment and machine-learning assessment of human body symmetry, provided herein is an adaptable, interpretable, end-to-end system for assessing infant symmetry from still images, with a view towards applications to the early detection and treatment of ASD, CMT, CP, and other common neurodevelopmental conditions.

### Pose-Based Symmetry Measurement

In this disclosure, a simple parameterized measurement of symmetry for body limbs based on 2D or 3D body joint locations is used. First, the infant 2D or 3D pose or skeleton, a collection of human joint locations, is extracted from a flat image by pose estimation algorithms. There are mature computer vision algorithms for this task, but their performance is weaker in the data-scarce infant domain. Accordingly, analysis and experimentation described herein used models adapted specifically to infant bodies.

For 2D pose extraction, the fine-tuned domain-adapted infant pose (FiDIP) model was used, which works by fine-tuning from an adult pose model to the infant domain, leveraging a domain adversarial network to learn equitably from both real and synthetic infant data. For 3D infant pose detection, a heuristic weakly supervised human pose (HW-HuP) estimation approach was applied. HW-HuP learns partial pose priors from public 3D human pose datasets in flexible modalities, such as RGB, depth or infrared signals, and then iteratively estimates the 3D human pose and shape in the target infant domain in an optimization and regression hybrid cycle. These infant 2D and 3D pose estimators can output 17 keypoints and 14 keypoint locations, respectively, but as described herein poses are restricted to the 12 body keypoints needed to define the upper and lower arms and legs (shoulders, elbows, wrists, hips, knees, and ankles), where asymmetry is most prominently manifested.

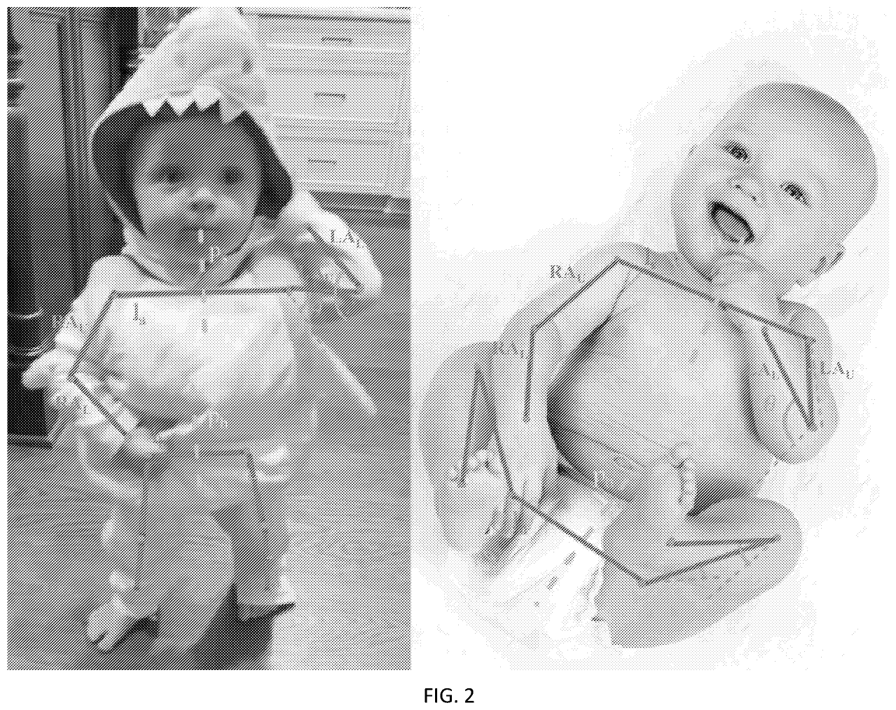

From the 12 keypoints in the body pose, measurements of angles and assessments of symmetry geometrically can be obtained as follows, and as illustrated in FIG. 2. First, consider the line segment lₛ connecting the two shoulder joints, and then define its mid-perpendicular pₛ, the line (in 2D) or plane (in 3D) which intersects lₛ orthogonally at its midpoint. Then reflect the upper right arm across pₛ, shift it so that its shoulder joint is aligned with that of the left upper arm, and measure the resulting angle. Similarly, reflect the right forearm across pₛ, shift it so that its elbow joint is aligned with that of the left forearm, and measure the angle. This is repeated for the legs: reflect, align, and compare the right versus left upper and lower leg angles, this time across the mid-perpendicular of the segment lₕ connecting the hip joints. If the formed angle of a given limb pair is less than some fixed predefined angle q, then the corresponding limb pair is considered to be symmetric, and otherwise it is asymmetric. By adopting the above proposed approach and varying angle thresholds, raw angle values and pose symmetry labels for each limb pair in infant images based on their 2D and 3D skeletons.

### Human Symmetry Assessment and Bayesian Aggregation

Pose asymmetry is often assessed by clinical experts to gauge neurodevelopment, or as a symptom for certain developmental disorders. To guide algorithmic efforts in emulating clinical evaluations, a number of human raters were surveyed for their assessments of pose angle differences and symmetry in infant images, for the pairs of limbs from the symmetry measurement above. The raters were asked to assess angle differences for limb pairs as per the measurement method, and also to make a subjective judgement of symmetry for each limb pair unguided by this method, to reduce redundancy and to capture information about innate symmetry assessments.

Through this survey, it was discovered that, in practice, there is large variation and weak agreement amongst assessments from human raters. Furthermore, this lack of reliability is not alleviated by simple majority voting, in part because such voting is susceptible to noise from outliers. To rectify this, a probabilistic approach is employed to evaluate different annotators and also give an estimate of the actual hidden labels. When multiple annotators provide possibly noisy labels and there is no absolute gold standard, a maximum-a-posteriori (MAP) estimator is proposed to jointly learn the classifier or regressor, the rater's accuracy, and the actual true label. The performance of each rater is measured by calculating sensitivity and specificity with respect to the unknown gold standard, and then a higher weight is assigned to them. An expectation maximization (EM) algorithm is applied to measure the performance of raters based on the given standard, and then optimize the standard based on the new rater performance. The gold standard is initialized by the majority voting result.

Based on those performance ratings, it is preferable to trust some particular raters more than others. Thus, a prior knowledge is imposed in the system to capture the skill of different raters. Beta priors, randomly initialized, are given as conditional information when calculating the probabilities of sensitivity, specificity, and prevalence for the Bayesian aggregation approach. Specifically, following the data gathered in the survey, there were two different types of rating labels: (1) binary class labels, as symmetric or asymmetric, and (2) angle class labels [<30°, 30°-59°, ≥60°], which intrinsically ordered. For the binary symmetry labels, the human true label is inferred directly following the EM optimization procedures mentioned above. For the angle class labels, the variable is binarized into one of two different ways, into classes [<30°, ≥30°] and [<60°, ≥60°], and then applying the same estimation procedure twice, once for each binarization. This yields separate probability estimates for the two binary classes, from which the most likely class can be inferred among [<30°, 30°-59°, ≥60°]. In some rare cases, where the two predicted probabilities are inconsistent with each other, the class with the highest formal probability is selected.

Annotation from Humans and Machines

In order to evaluate the performances of human rating and pose-based symmetry measurement, they were applied to a real infant image set of the publicly released synthetic and real infant pose (SyRIP) data, which contains 700 real images with assigned posture labels (supine, prone, sitting, and standing) and annotated 2D keypoint locations. See Table 1, included as FIG. 16, for an overview of the data types and sources discussed herein.

### Human Symmetry Survey

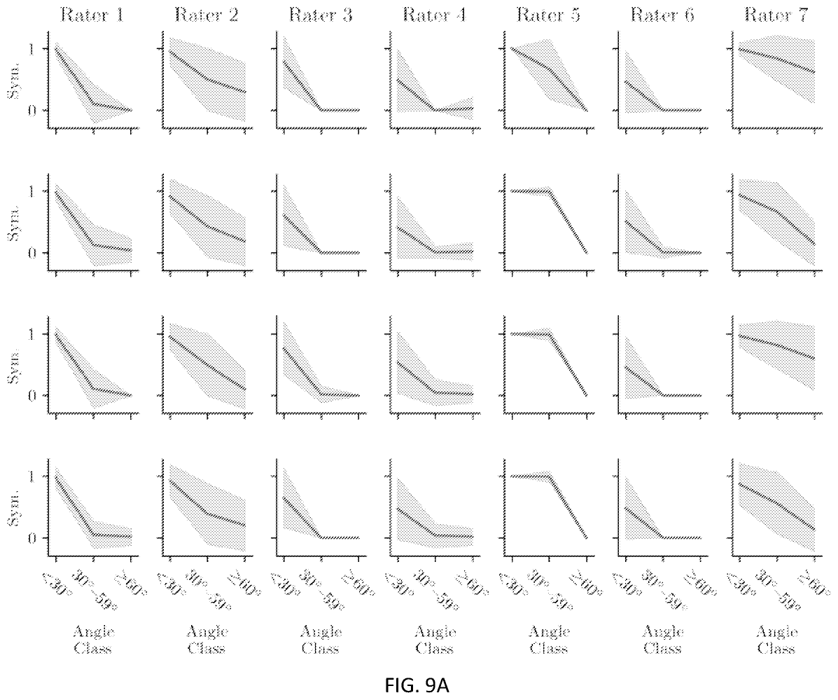

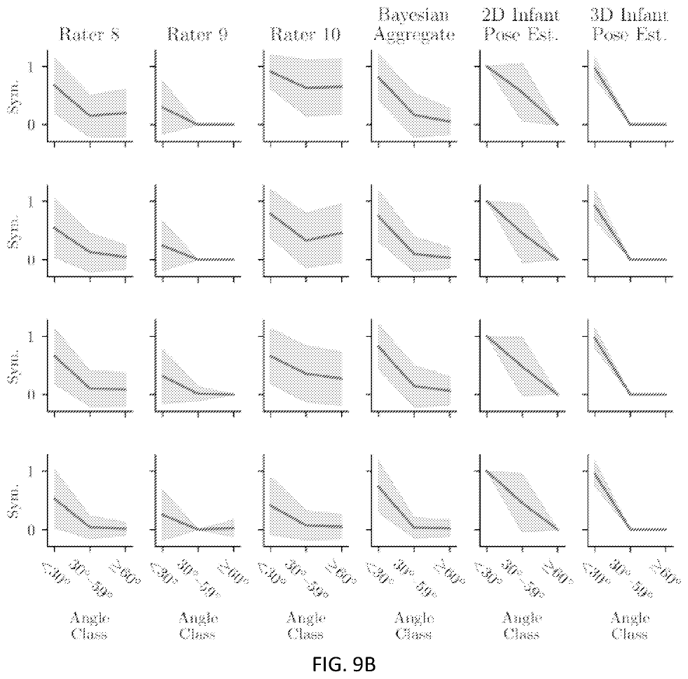

In order to reveal and simulate the mechanism of human rating for postural symmetry, an online experiment study was conducted to collect the pose symmetry judgement responses of SyRIP real images from 10 raters through a Qualtrics platform. The 700 images were divided into 28 blocks, each of which had 25 questions. The questions in each block were randomly assigned to each participant. There were two sessions of mandatory resting time (5 minutes) assigned after the 10th and 20th blocks. Each image was accompanied by eight questions: four of them regarding the symmetry of the four limb pairs (upper arm, lower arm, upper leg, and lower leg) and the rest about the predicted angle class between each of the four pairs of limbs. There were five demographic questions at the end of the survey about their major, gender, age, education level, and experience in computer vision or drawing (23 was the mean age; there were 5 male and 5 female participants). A basic snapshot of the survey responses, which plots the mean rater assessment of symmetry at each assessed angle class, can be found in FIGS. 9A and 9B.

### Infant 2D and 3D Pose Estimation

The performance from a number of pose estimation models was tested and is listed in Table 2.

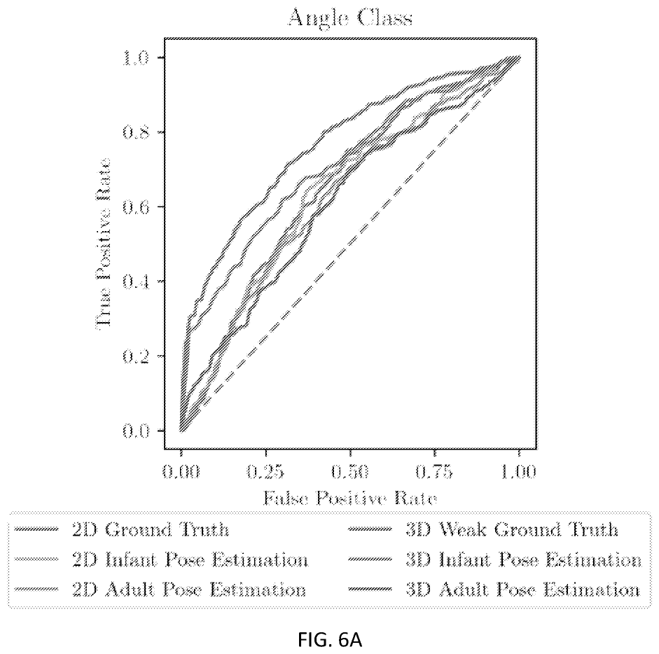

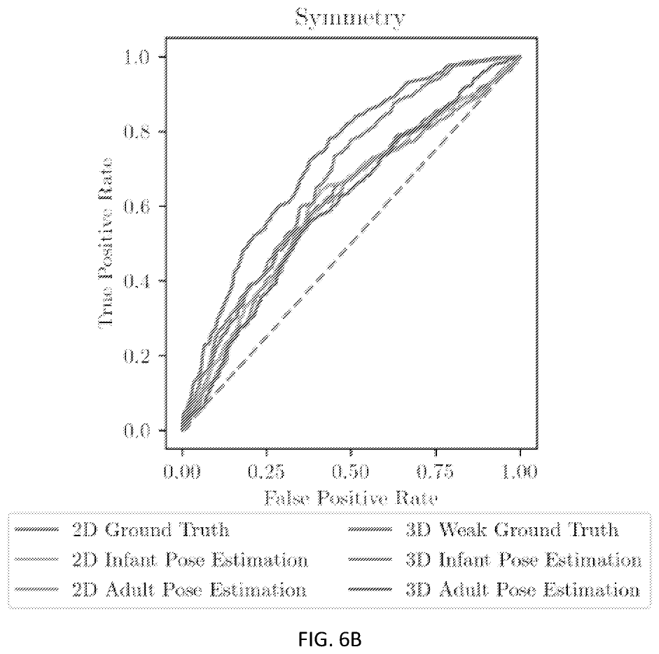

TABLE 2
At top: pose estimation models or ground truth data, from which
symmetry assessments are derived. At middle: optimal threshold
angle for each pose estimation model for obtaining symmetry
assessments with the highest Cohen's κ agreement with
the Bayesian aggregated symmetry assessments. At bottom: Areas
under the receiver operating curves (FIGS. 6A-6B) for logistic
regression of the Bayesian aggregated symmetry assessment using
the raw angles obtained from each pose estimation model.

| | | | Adult Pose | Infant Pose | Ground |
|---|---|---|---|---|---|
| | | | Est. | Est. | Truth |
| Source | | 2D | Dark Pose | FiDIP | SyRIP |
| | {open oversize brace} | 3D | SPIN | HW-HuP | Corrected |
| | | | | | HW-HuP |
| Opt. Angle | | 2D | 43.7 | 42.0 | 39.1 |
| | {open oversize brace} | | | | |
| | | 3D | 17.8 | 27.9 | 27.7 |
| ROC AUC | | 2D | 0.60 | 0.61 | 0.62 |
| | {open oversize brace} | | | | |
| | | 3D | 0.60 | 0.68 | 0.73 |

The DarkPose model, which is trained on large-scale public human pose datasets, was applied as adapted to infant poses using the FiDTP model to predict 2D keypoints for 2D pose-based symmetry measurement. The well-performed human 3D pose estimation model, SPIN, and the infant-adapted 3D pose estimator, HW-HuP, were used to infer 3D keypoints for the described 3D pose-based symmetry assessment. 2D and 3D pose ground truth come from the SyRIP dataset and corrected HW-HuP predictions, respectively.

### Infant 3D Pose Correction

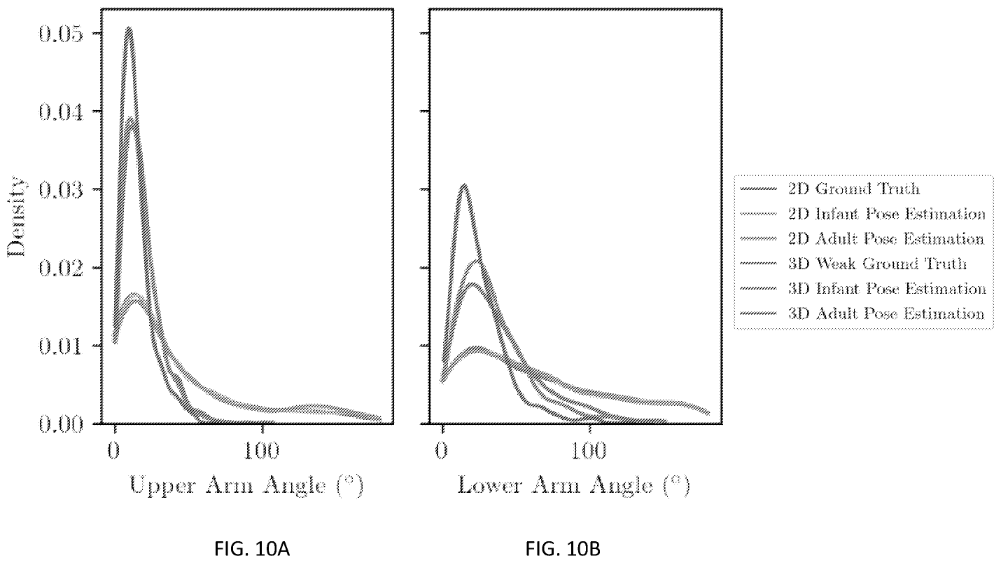

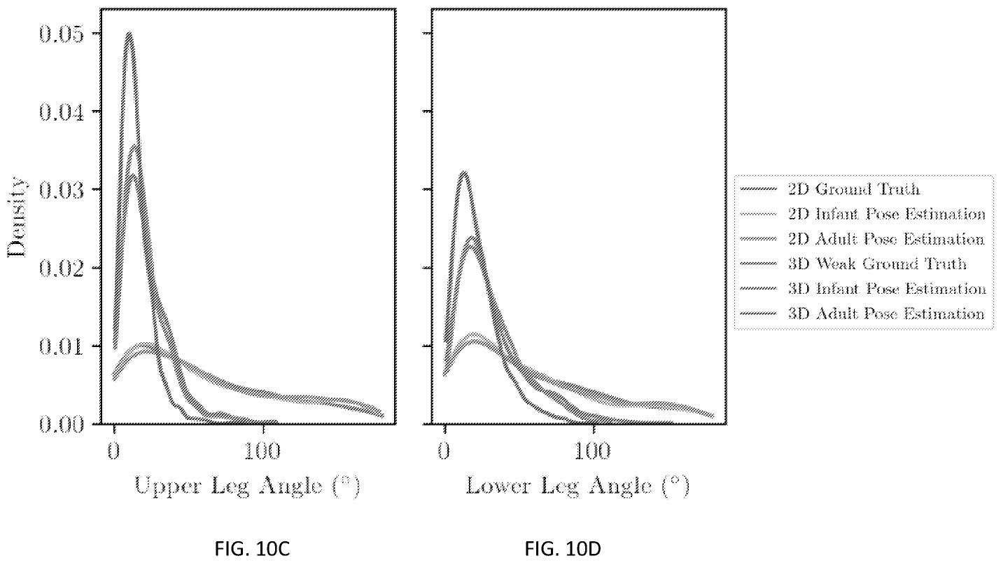

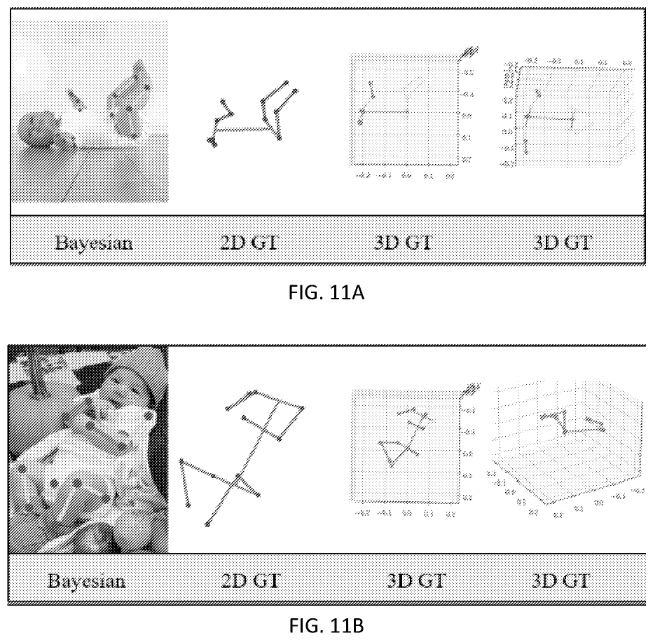

The performance of the pose-based symmetry assessment depends largely on the accuracy of the 2D or 3D pose estimation. The SyRTP dataset, however, only contains ground truth keypoint locations in 2D coordinates, not 3D. So far there does not exist a robust 3D pose estimation model to provide satisfied infant 3D pose inference. Therefore, to cope with this unreliability, an interactive annotation tool was modified to correct poses predicted by the infant 3D pose estimator, HW-HuP. Because this pose estimator also estimates camera parameters, its 3D pose keypoint predictions were overlaid onto the 2D plane over the original infant image, to ensure 2D pose alignment. The global pose orientation and the local bone vector orientation of the 3D skeleton were interactively modified by keyboard inputs to make both the 3D skeleton and the real-time updated projected 2D keypoints locations as correct as possible. In this way, a weak ground truth of 3D pose was obtained. This is considered a weak ground truth because of inevitable error associated with human vision and camera parameter estimation. The distributions of predicted angle differences obtained from various 2D and 3D pose estimation models or ground truth are exhibited in FIGS. 10A-10D.

### Analysis: Computer Vision to the Rescue

The analysis that follows begins with an examination of the shortcomings of human ratings of symmetry and illustrates how the described Bayesian aggregation process ameliorates some of these issues. The ability of the 3D infant pose estimation system is then demonstrated with respect to predicting the Bayesian aggregate assessments of angle and symmetry to a higher degree than adult or 2D pose-based models, illustrating the effectiveness of both the Bayesian aggregates and the 3D pose estimations. Then, end-to-end symmetry assessments are produced by calibrating the 3D pose-based symmetry assessments with the Bayesian aggregate data.

Performance gains afforded by the 3D infant pose-based system over 2D or adult pose-based alternatives are discussed to illustrate the advantages offered by the described methods and systems for computer vision-based assessment of infant face and body symmetry over the human and even Bayesian aggregate assessments, both quantitatively and qualitatively. In addition, further description and explanation with respect to 3D pose estimation and factors affecting symmetry assessment is provided.

### Amending Incongruent Human Annotations

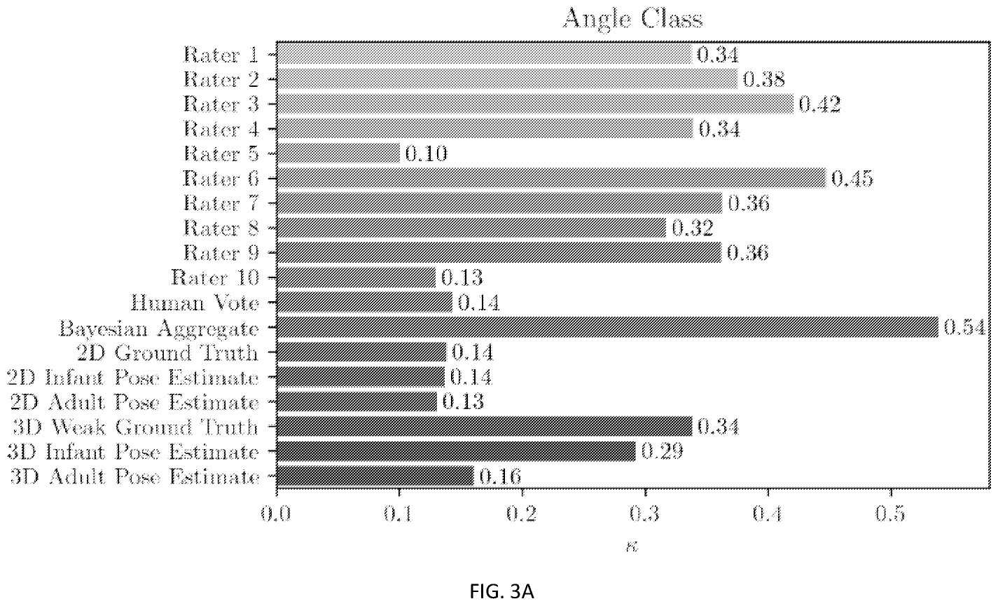

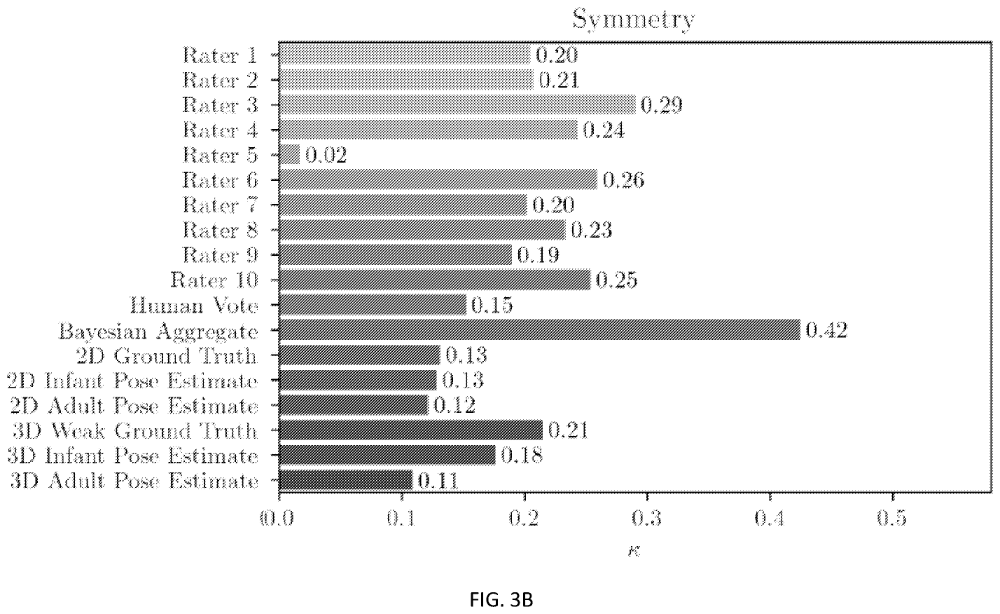

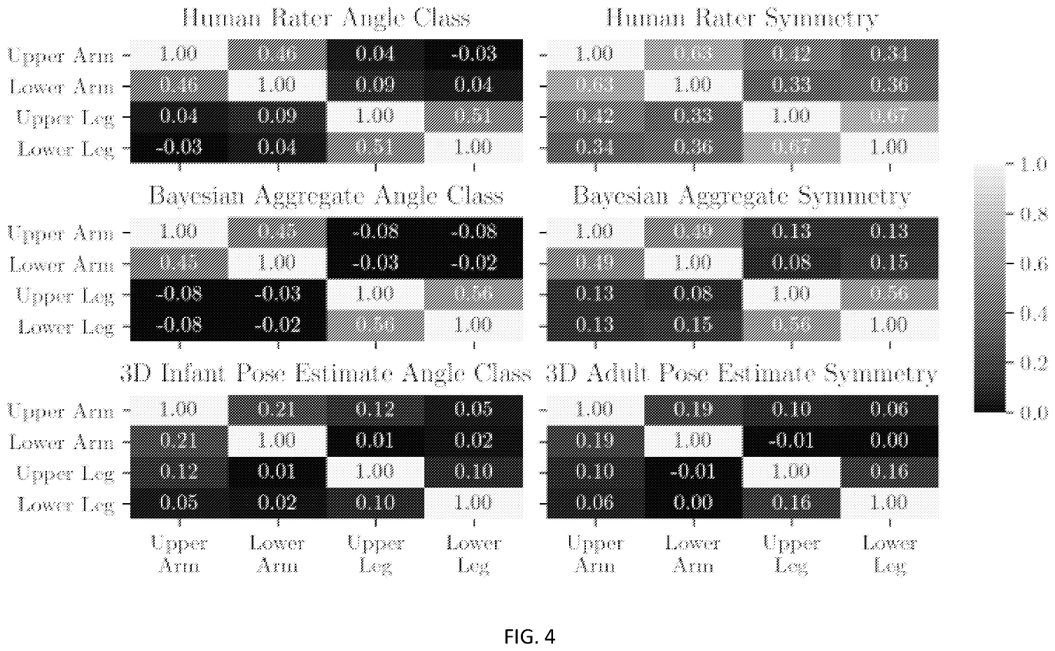

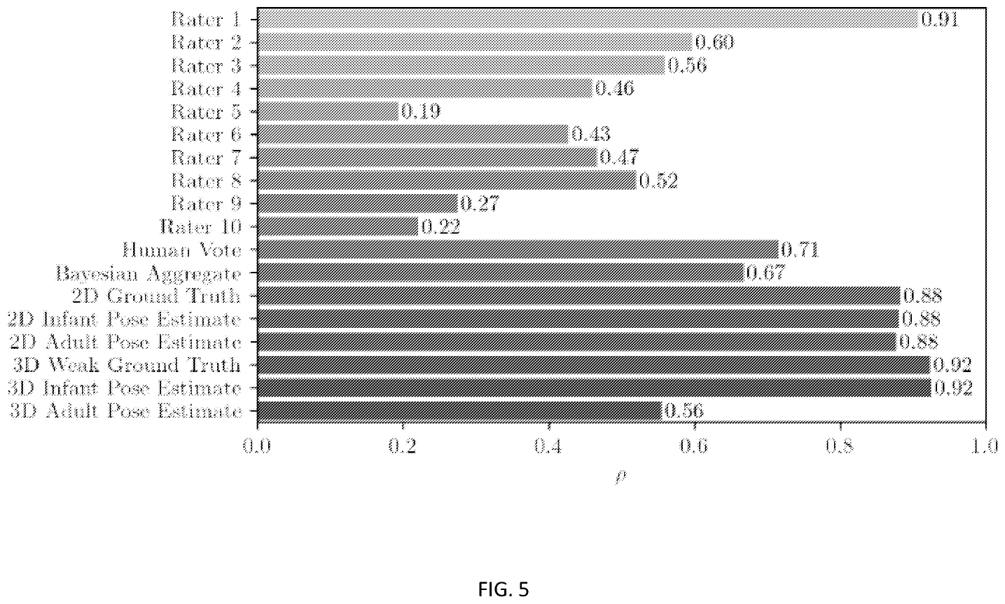

The average Cohen's κ agreement between each human rater and their other nine fellow human raters, on their assessments of angle class and symmetry across four pairs of limbs and 700 real images in the SyRIP infant dataset, can be found in FIGS. 3A-3B. These illustrate average agreement for angle class assessments characterizable as “fair” and average agreement for symmetry assessments characterizable as “slight” to “fair”. In the same vein, the Krippendorffs a collective agreement amongst the entire group of human raters is 0.30 for angle class and 0.18 for symmetry, illustrating collective agreement characterizable as “fair” and “poor”. In addition to lower inter-rater agreement, human assessments are also afflicted with high inter-limb assessments agreement for angle class and especially symmetry, as seen in FIG. 4. The high arm-to-leg agreement in symmetry assessments in particular likely indicate unwarranted bias, given that corresponding arm-to-leg agreement for angle class are negligible. FIG. 5 illustrates that human ratings of angle class exhibit a low level of correspondence with human ratings of symmetry, suggesting a low level of internal consistency among individual human ratings.

Underlying many of these issues is the high variance in assessments between raters (illustrated starkly in the summary of individual rater responses illustrated in FIGS. 9A-9B) and the high variance of the resulting agreement and consistency metrics. These issues led to the use of methods of aggregating the human ratings into a more cohesive whole, including a simple voting method and the probabilistic Bayesian aggregation method described above.

The results corresponding to these aggregation methods (shown in FIGS. 3A-3B, FIG. 4, and FIG. 5) show that the Bayesian aggregate in particular enjoys lower inter-limb agreement and higher angle-asymmetry correspondence than the average human rater, suggesting respectively, lower levels of bias and higher internal consistency, all while maintaining a high level of agreement with the average human rater. Thus, the Bayesian aggregate assessment was adopted as a weak ground truth representation of human assessment of symmetry, with potentially undesirable characteristics excised. Table 3 reports performance metrics of individual human assessments of symmetry, relative to the Bayesian aggregate results as ground truth, again demonstrating the wide variance in human reliability.

TABLE 3
Performance of each human rater assessment
of symmetry relative to Bayesian aggregate.

| Rater # | 1 | 2 | 3 | 4 | 5 | 6 | 7 | 8 | 9 | 10 |
|---|---|---|---|---|---|---|---|---|---|---|
| Sensitivity | 0.98 | 0.97 | 0.88 | 0.54 | 0.99 | 0.61 | 0.99 | 0.64 | 0.38 | 0.79 |
| Specificty | 0.35 | 0.38 | 0.80 | 0.96 | 0.03 | 0.96 | 0.30 | 0.89 | 0.98 | 0.80 |

### Pose-Based Symmetry Assessment

Analysis of the extent to which the pose-based systems can track the Bayesian aggregate symmetry assessments is described below, followed by calibration and evaluation of the computer vision based infant symmetry assessment system.

The raw angle data obtained from pose estimation, described above, includes a set of four angle differences, in degrees, for the four key pairs of limbs under consideration (upper arms, lower arms, upper legs, and lower legs). Such data is eventually converted to the discrete signal of the angle category and symmetry assessment, but, initially, the maximum amount of information is retained and gauge agreement with the Bayesian aggregate rater assessments of angle class and symmetry for each of the four joints and each infant image (2800 data points in all) is determined by logistically regressing for them using the raw angles. FIGS. 6A-6B show the receiver operating characteristic (ROC) curves resulting from this regression, performed with a 3:1 train-test split. For the regression of angle class, the three true classes are compressed into a binary variable indicating whether the angle is over 30′ for ease of interpretation. The areas under the curve (AUC) for the ROC curves in the symmetry regression are provided in Table 2.

These metrics confirm that the raw angles from the weak 3D ground truth can model the Bayesian aggregate assessment of both angle and symmetry to a high degree of fidelity. Neither set of data can be taken as fully reliable ground truth, but since they are derived from different human annotators performing fairly different tasks, the high level of agreement exhibited here increases confidence in the accuracy of both. Among pose estimation models, the 3D infant-specific models enable the next best predictions of human symmetry assessments, while the poses from the remaining models, either 3D pose models for the general most adult human, or 2D pose models for infants or adults, offer weaker ability to predict the human assessments.

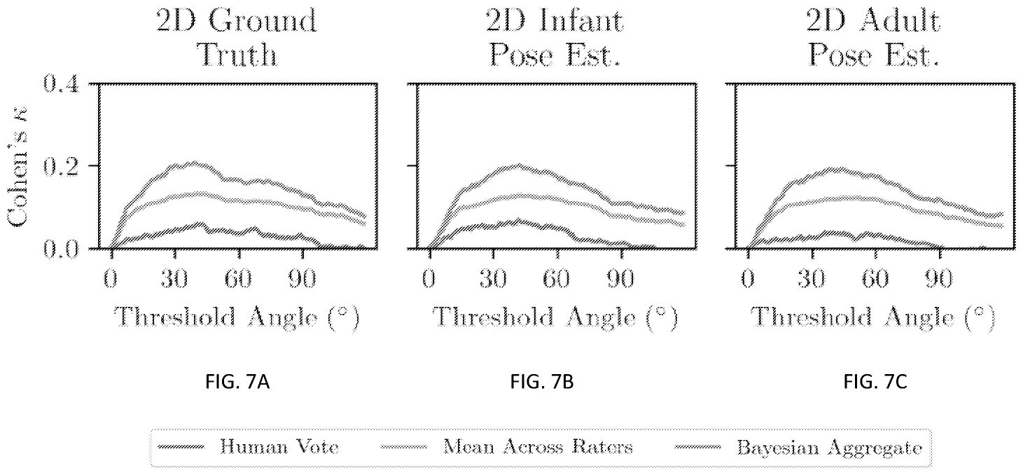

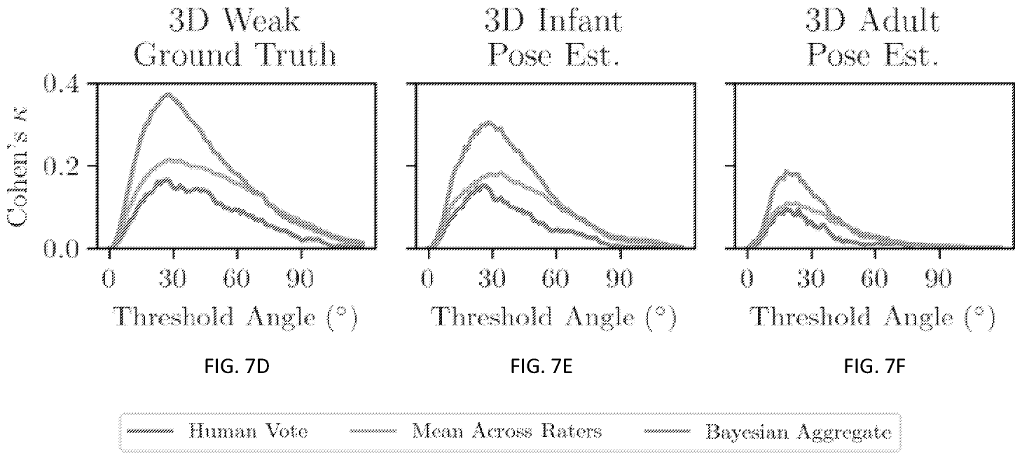

Calibrating an end-to-end pose-based system, the computer vision based infant symmetry assessment system, was undertaken for the evaluation of symmetry for use in practical applications or further research. In concrete terms, it is desirable to select threshold angles which will allow conversion of raw joint angles to binary assessments of symmetry per joint, in a way that maximizes concordance with the Bayesian aggregate. Guidance of the concordance was achieved with the same Cohen's κ agreement score discussed above. FIGS. 7A-7F show the Cohen's κ agreement of symmetry assessments derived from all six of the pose-based estimators at various decision angle thresholds, compared with both the voted and Bayesian raters; it also shows the mean Cohen's κ agreement with each of the ten human raters individually. Incidentally, these results not only confirm again the supremacy of the 3D ground truth and infant pose prediction methods for tracking human assessments of symmetry, but on the flip side, also demonstrate the superiority of the Bayesian aggregation of human symmetry assessments over the voted or the average human assessment for tracking the 3D weak ground truth assessment, at most reasonable angle thresholds. From these Cohen's κ curves, the threshold angles for maximizing agreement for each pose-based model are extracted, reported in Table 2. These thresholds then define the corresponding symmetry assessment for each model (or ground truth data).

Metrics quantifying these final assessment models have already been reported throughout this disclosure and are further interpreted here. FIGS. 3A-3B, confirm, as expected, that the 3D infant pose estimation assessment offers the highest average agreement with human rater assessment, compared to the 3D adult pose estimation or the 2D infant pose estimation models. The 3D infant pose estimation assessment also comes close to the level achieved by the 3D weak ground truth assessment. As shown in FIG. 4, assessments based on predicted or weak ground truth 3D poses are relatively free from inter-limb agreement, compared to individual or aggregate human assessments. In the absence of fully reliable ground truth assessments, this circumstantially suggests that human assessments are susceptible to bias from nearby parts, while the automated approach is not. FIG. 5 shows that most of the pose estimation-based models enjoy high internal consistency in their assessment of angle class versus symmetry, as to be expected from mechanistic models.

### Qualitative Evaluation

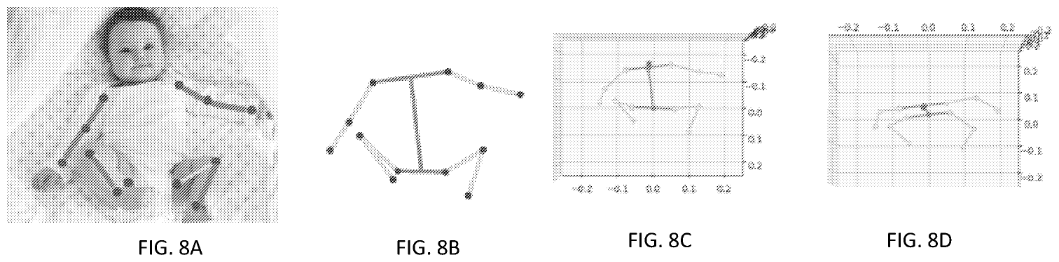

The performance of the pose-based models illustrated in FIGS. 1A-1H and FIGS. 8A-8D were evaluated. Those figures FIGS. 1A-1H and FIGS. 8A-8D each illustrate the Bayesian aggregate assessments of symmetry on top of the original image, as well as the assessments derived from the 2D and 3D infant pose models (FiDIP and HW-HuP, respectively) on top of their respective predicted pose skeletons, with green indicating symmetric judgements and red indicating asymmetric judgements.

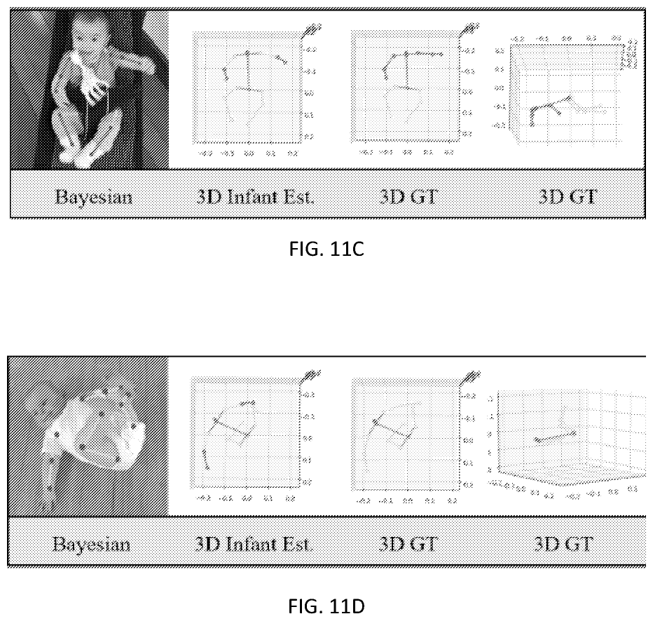

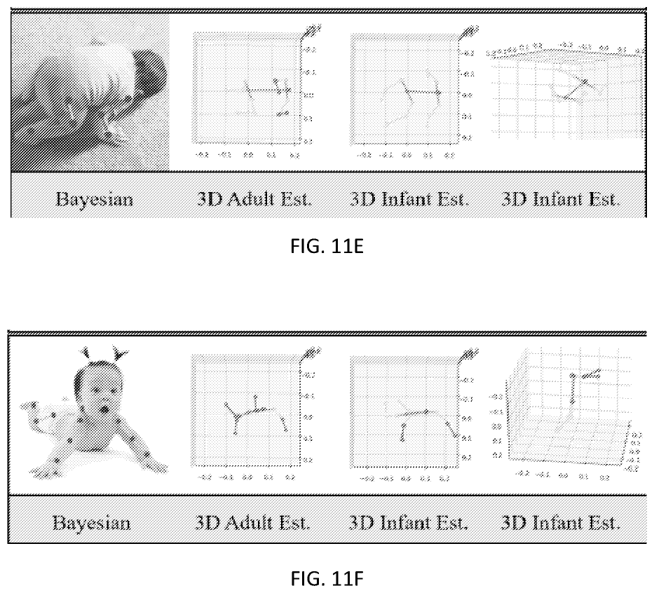

In FIGS. 1A-1H, examples are shown of infant poses where the 2D pose-based assessment is mistaken, but the 3D pose-based assessment is able to make the correct call, according to the Bayesian aggregate label. Multiple views of the 3D skeleton are illustrated to highlight the advantage that the 3D pose-based assessment has and suggest that the mistakes made by the 2D pose-based assessment are expected, given that 2D is limited to a single perspective. FIGS. 8A-8D show a special case where limits on the 2D perspective are mitigated because the infant is lying flat on its back with its limbs largely confined to a plane parallel to the image plane. Indeed, in this case, the 2D and 3D pose-based symmetry assessments agree, although both differ from the Bayesian assessment, which may reflect human bias or simply a stricter subjective threshold for symmetry on the part of the human assessments. More comparisons between 2D or 3D pose-based assessments are illustrated in FIGS. 11A-11F.

### Improving 3D Pose Estimation

The main factor limiting performance of state-of-the-art infant 3D pose estimators such as HW-HuP is the scarcity of “true” ground truth 3D pose data. Thus, as explained herein, there are beneficial effects of fine-tuning HW-HuP with the weak 3D ground truth labels for SyRIP images generated for this disclosure. The 700 SyRIP images were split into a 100-image test set, coinciding with SyRIP's Test100 set, and a 600-image train set. The infant HW-HuP model was then fine-tuned on the 600-image train set with weak 3D labels, for 200 epochs with learning rate of 5e-05. The resulting performance of the fine-tuned infant HW-HuP model under the mean per joint position error (MPJPE), as reported in Table 4, is significantly improved over the base infant HW-HuP model, and over the adult pose SPIN model.

TABLE 4
Reports 3D pose estimation performance in mean per joint
position error (MPJPE) in mm on the 100 image SyRIP test
set with the weak ground truth 3D labels. The fine-tuned
HW-HuP model (HW-HuPFT) is compared with the base HW-
HuP model, and with the adult-trained SPIN model.

| Model | SPIN | HW-HuP | HW-HuP-FT |
|---|---|---|---|
| MPJPE | 105.8 | 97.2 | 78.3 |

### Factors Affecting Symmetry Assessment

A supplementary importance analysis of factors affecting the assessments of symmetry was performed via logistic regression. The Bayesian aggregate symmetry assessment was taken as the response variable, and the following were taken as covariate factors: limb part under consideration (upper arm, lower arm, upper leg, or lower leg), the infant posture (included in SyRIP), an occlusion label for each limb (which is annotated for this purpose), and finally, an angle variable. Two separate sources were used for the angle variable, one including the angle class assessments from all of the human raters, and one obtained from the 3D infant pose estimation. That analysis shows that the regressed models are statistically significant.

According to the logistic regression result for Bayesian aggregation, all four predictors account for between 40.8% (R²_{CS}) and 54.6% (R²_{N}) of the variance in the dependent variable and correctly classify 83.2% of cases. From the logistic regression result for four predictors, it is concluded that only limb part and estimated angle between corresponding limb part significantly contributed to the asymmetry assessment model. For the 3D prediction model, the logistic regression result indicated that all factors except occlusion significantly contribute to the model. All four factors explain between 45.5% (R²_{CS}) and 60.5% (R²_{N}) of the variance in the dependent variable and correctly classify 90.8% of cases.

In order to assess predictor importance, the decrease of R²_{CS} approach was used to calculate the ΔR²_{CS} when removing one of the predictor. A larger decrease indicates more contribution of the removed predictor to explain the model. The results of decreasing R²_{CS} are reported in Table 5. When the angle estimation was taken out of the model, the R²_{CS} value declined by −0.342 for the Bayesian model and by −0.366 for the 3D prediction model correspondingly. Thus, angle estimate is the most important predictor of all the variables that have been found, and it is used in both the human rating model and the 3D prediction model.

TABLE 5
Parameter importance evaluation for logistic regression
of Bayesian aggregate symmetry class from human rater
and 3D infant pose estimate assessments.

| | |
|---|---|
| Human Raters | 3D Infant Pose Est. |

| Excluded Feature | R_{CS}² | ΔR_{CS}² | R_{CS}² | ΔR_{CS}² |
|---|---|---|---|---|
| None (Full Model) | 0.408 | | 0.455 | |
| Limb Part | 0.402 | −0.006 | 0.418 | −0.037 |
| Posture | 0.408 | 0.000 | 0.448 | −0.007 |
| Angle | 0.066 | −0.342 | 0.089 | −0.366 |
| Occlusion | 0.408 | 0.000 | 0.455 | 0.000 |

Table 6 reports the results of an additional regression variance analysis. These results further reinforce findings regarding the advantages of 3D pose-based symmetry assessment over both human ratings (individual or in aggregate) and 2D pose-based systems.

TABLE 6
Proportion of variance R2 values from three linear regression models,
all with the 3D weak ground truth raw angle as the dependent variable
(and each of four pairs of limbs across 700 infants as the sample
space). All three models include the labels for the limb part, the
posture, and occlusion as independent variables, together with an
angle class label drawn respectively the 2D infant pose estimation,
3D infant pose estimation, or the Bayesian aggregate assessment.
The resulting R2 and adjusted R2 scores can be interpreted as gauging
the predictive power of each respective model, with our 3D infant
pose estimation method holding a clear advantage.

| Model | R² | Adjusted R² |
|---|---|---|
| Bayesian Aggregate | 0.365 | 0.360 |
| 2D Infant Pose Estimation | 0.229 | 0.223 |
| 3D Infant Pose Estimation | 0.496 | 0.492 |

Exemplary Applications of Computer Vision Based Assessment of Postural Symmetry A computer vision-based method for assessment of postural symmetry in infants from their images is described hereinabove, with the goal of enabling early detection and timely treatment of issues related to infant motor and neural development. Human ratings of symmetry were found to be unreliable, and methods for rectifying such ratings with a Bayesian-based probabilistic aggregate rating were also described. Automatic assessments based on pose estimation were shown to avoid some of the pitfalls of human assessments, while retaining the ability to predict the Bayesian aggregate ratings to a strong degree, with 3D infant pose models performing stronger than 2D models or adult pose models. The potential application of such systems and methods to screening, diagnosis, and monitoring of Torticollis is described below.

### Application of Computer Vision to Torticollis

Herein, computer vision pose estimation techniques developed expressly for the data-scarce infant domain (e.g., the methods and systems for computer vision-based assessment of infant face and body symmetry described above), are applied to the study of torticollis, a common condition in infants for which early identification and treatment is critical. Specifically, a combination of facial landmark and body joint estimation techniques designed for infants are used to estimate a range of geometric measures pertaining to face and upper body symmetry, drawn from an array of sources in the physical therapy and ophthalmology research literature in torticollis. Performance is gauged using a range of metrics, showing that the estimates for most of the geometric measures are successful, yielding very strong to strong Spearman's ρ correlation with ground truth values. Furthermore, it is demonstrated that these estimates derived from pose estimation neural networks designed for the infant domain cleanly outperform estimates derived from more widely known networks designed for the adult domain.

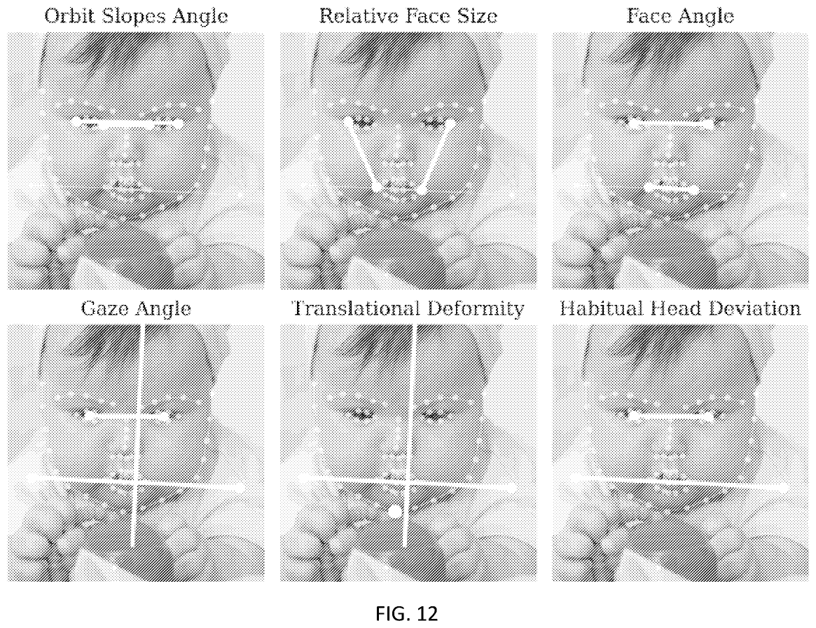

The geometric symmetry measures used herein are illustrated in FIG. 12. These geometric symmetry measures were carefully researched and selected from among those studied by physical therapy and ophthalmology researchers to enable qualitative assessment of torticollis over time and in response to treatments, including surgery. That research included studying and considering the reliability of the procedure of extracting such measurements from still photography using computer vision pose estimation techniques. Results show that the measurements from individual still photographs can be employed, alongside other tools, as part of detection and treatment of torticollis conditions.

The technical tools used for this proof-of-concept experiment are based in computer vision pose estimation from still images and, in particular, face and body landmark estimation. Mature solutions to these tasks exist but are generally based in deep learning from primarily adult faces and bodies. As explained above, specialized methods tailored for the unique faces and bodies of infants have only begun to crop up in recent years, in recognition of the significant domain gap between infant and adults from the point of view of computer vision representations. In this experiment, an infant face landmark estimation model was used together with an infant body landmark estimation model, both of which employ domain adaptation techniques to tune existing adult-focused models to the infant domain. A subset of the InfAnFace Test set was used, and the values of the six geometric symmetry measures derived from both predicted and ground truth landmarks were compared. In addition, these models were modified to enable compatibility with the landmarks used in pose estimation techniques.

The findings show that predictions derived from infant-domain pose models exhibit “strong” or “very strong” Spearman's ρ ranked correlation with the ground truth values on a precisely labeled test set of infant faces in the wild, with best performance on the gaze angle (ga) between the line connecting the outer corners of the eyes and the midsternal plumb line and the habitual head deviation (hhd), the angle between the eye line and the acromion process (shoulder) line. Predictions of three other measurements (including non-angles) were strong, but only moderate success was found in the predictions of the orbit slopes angle (osa), the angle between the lines connecting the outer and inner corners of the eyes, arguably the most subtle metric. Based on these findings and analysis involving further performance metrics below, it is clear that computer vision infant pose estimation techniques can successfully measure a range of quantities pertaining to torticollis.

### Quantifying Torticollis

Previous research and methodology in the diagnosis and treatment of torticollis is largely based on in-person physical assessments by experts and follow-ups with imaging or other deeper techniques. By contrast, the present technology deals with measuring signs and symptoms of torticollis geometrically from still images.

With respect to congenital muscular torticollis (CMT), the effectiveness of a specific therapeutic intervention for CMT was studied by comparing changes in an infant's head tilt, as measured by hand from still photographs, as was the reliability of this still photograph assessment method itself. Separately, the “gaze angle” and “transformational deformity” of child subjects were measured, again from still photographs, to gauge improvement in response to surgical intervention. Measurements from still photography offer researchers a somewhat repeatable, objective way to quantify the change in severity of torticollis before and after an intervention, although, as noted above, human raters are often inconsistent and biased.

Sometimes torticollis is not congenital (present from birth) but rather acquired, as is the case with ocular torticollis, where the abnormal head posture is adopted to compensate for a defect in vision. In such cases, diagnosis often only occurs in adulthood and can be informed by examination of head pose in childhood photographs. Correspondingly, ophthalmologists have also studied the quantification of facial asymmetry via geometric measurements from still images. Such methods rely on facial measurements such as the “orbit slopes angle,” “relative face size,” “facial bulk mass,” “facial angle,” and the “nasal tip deviation.” These measurements are studied not as a means to quantify the effect of interventions, but as part of a more comprehensive study on the differential diagnosis of ocular torticollis and other conditions related to facial asymmetries, especially superior oblique palsy.

### Computer Vision to Monitor Infant Health and Development

Researchers have employed computer vision to analyze head posture and tremor in the context of algorithmic understanding of cervical dystonia (also known as spasmodic torticollis), with incidence largely in the adult population. Accordingly, those studies can take advantage of far more mature adult-focused head pose estimation techniques like OpenFace 2.0, whereas the present efforts are highly constrained by data scarcity in the infant domain. In the infant domain, there is some prior work on bodies as discussed above, but not faces. Some prior systems use an infant-specific body pose estimation deep network to extract body motion information from infant videos, in an attempt to assess infant neuromotor risk. Nevertheless, the inventors are not aware of prior computer vision research intended to detect torticollis or gauge head asymmetry in infants.

### Measures of Geometric Symmetry

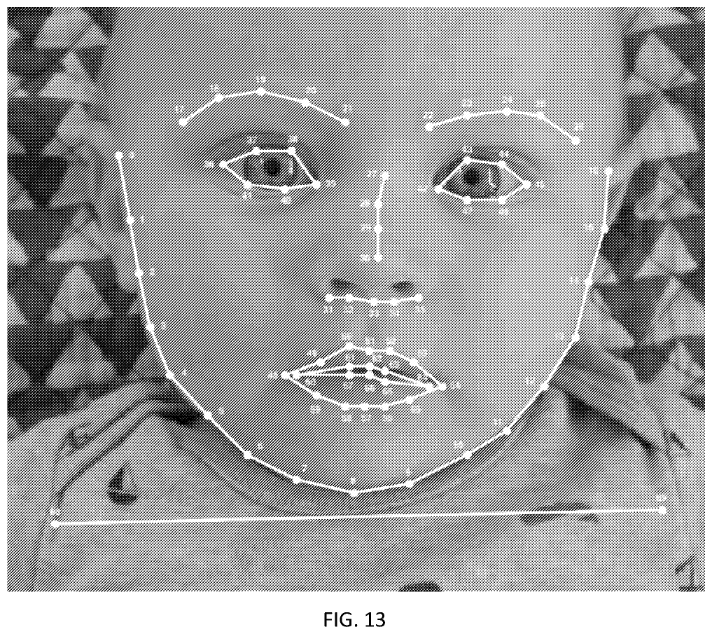

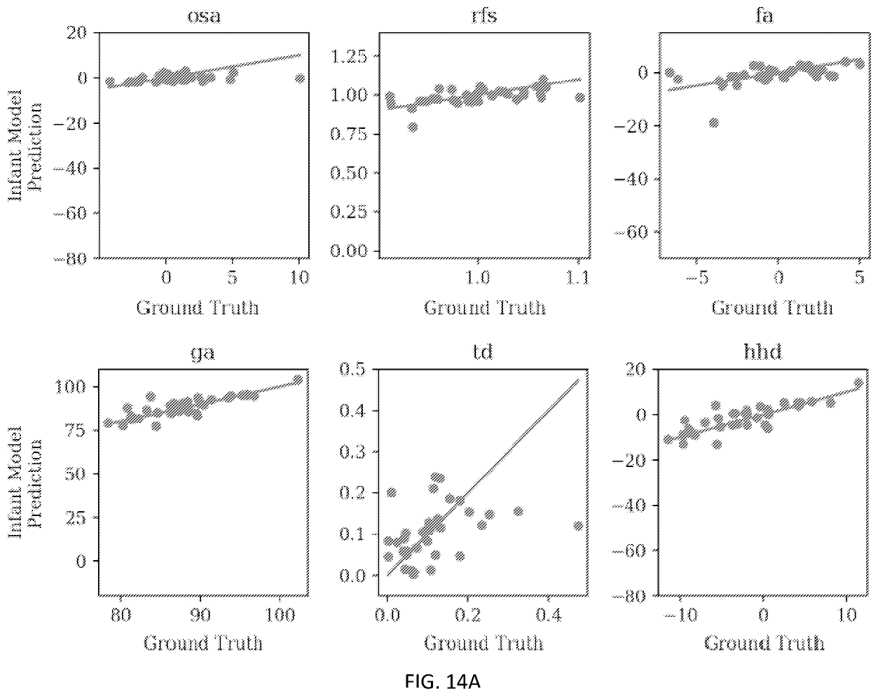

As noted above, all clearly defined geometric symmetry measures used by researchers in the study of torticollis and facial asymmetries were selected, six in all. The definitions were then altered to base them explicitly on the 68 facial landmark coordinates and two body joint (shoulder) coordinates used by the pose estimators described herein, as illustrated and enumerated in FIG. 13, and to enable more consistent comparisons. The final geometric symmetry measures, described in more detail below, are defined in Table 7 (FIG. 17) and illustrated in FIG. 12.

### Assumptions and Context

Because both ground truth and estimated landmark coordinates are used in two dimensions, it is assumed that every infant is facing forward into the camera, so that the infant's face plane is roughly parallel with the camera image plane. In principle, all of the considered measurements are well-defined for three-dimensional face and bodies, but there is no access to three-dimensional face landmarks in the infant domain and thus this alignment must be assumed to ensure that these measurements are well-defined from the two-dimensional landmarks.

### Symbols and Functions

In Table 1 (FIG. 16), [symbol] (u, v) represents the signed angle in degrees between vectors u and v, relative to a fixed orientation (say, clockwise). It is preferred to measure the signed angle because, for instance, it is preferred to have a clockwise and counterclockwise angles of equal magnitude to be considered different, for the purposes of quantifying predictions. Note that [symbol] (u, v)= [symbol] (v,u) for all u and v. Furthermore, Pᵢ denotes the ith landmark (with i∈{0, . . . , 67} corresponding to facial landmarks and i∈{68, 69} the shoulders); \vec{PQ} denotes the vector between two points P and Q,

$${P}_{i , j}^{μ}$$

is the midpoint between Pᵢ and Pⱼ; ⊥ᵤ is the perpendicular vector to u (say, taken clockwise); ∥·∥ is the L² norm; and ∥P,u∥ is the L² distance between a point P and the line spanned by a vector u. (Distances can are computed in pixels, but the final geometric measures based on distance are unitless because they are normalized, as described below.

### Definitional Choices

The descriptions in Table 7 (FIG. 17) were adapted for clarity and uniformity. These descriptions were then formalized into the geometric definitions in Table 7 (FIG. 17) by assigning face and body features to landmarks used in the described models (as in FIG. 13), with choices in specific cases as follows:
For facial angle (fa) and habitual head deviation (hhd), the eye line is interpreted as being the line between the midpoints of the eyes, as defined for each eye by the midpoint of its corners, to ensure consistency regardless of whether the eye is open or closed.
For relative face size (rfs), typically defined as the greater of the two outer-cathnus-to-mouth-corner lengths divided by the lesser, instead is defined here by removing the extra logical qualifier so as to always divide the left length by the right length to enable more precise evaluation of predictions.
For gaze angle (ga) and translational deformity (td), the perpendicular bisector is taken to the two shoulder joints as the midsternal plumb line.
For translational deformity (td), since there are no standardized photograph sizes, td is normalized by a relatively stable quantity, the distance between the outer cathnuses (eye corners).

### Omissions

The two following typical measurements were omitted. First, the “facial bulk mass” of one side compared to the other is not precisely defined and is perhaps not intended to be inferred from photographs. Second, the “nasal tip deviation” is also not made fully precise and was deemed too difficult to model reliably. Note that quantities were not excluded simply because they cannot be accurately measured algorithmically and indeed, as discussed below, it was found that the orbit slopes angle (osa) cannot be.

### Experimental Setup: Infant Data and Pose Estimation

### Selecting and Annotating Infant Faces

The infant annotated faces (InfAnFace) dataset, a comprehensive dataset of 410 infant faces labeled with 68 facial landmark coordinates and four binary pose attributes, designed specifically to alleviate the shortage of annotated data in the infant face domain, was selected as the testbed for the study. The images from the InfAnFace are captured from internet image and video sources and represent a diverse range of infants in natural environments “in the wild.” A subset of images is manually selected, the subset satisfying the requirements described herein that the infant be fully front facing, leaving 36 images from InfAnFace Test to work with. The ground truth facial landmark labels were manually augmented with two additional labels for shoulder joints, to enable application of the definitions in Table 7 (FIG. 17).

### Face and Body Landmark Estimation

To study the effectiveness of algorithmic assessment of the geometric measures, experiments were performed with two sets of pose estimation models, the first established models trained largely on adult data, and the second recent models designed specifically for the infant domain. The models and training data are summarized in Table 8.

TABLE 8
CONFIGURATION OF OUR LANDMARK
ESTIMATION MODELS

| Model | Task | Backbone | Training Data |
|---|---|---|---|
| HRNet [21] | Adult Face | HRNet | 300-W Train [16] |
| DarkPose [23] | Adult Body | HRNet | COCO [12] |
| HRNet-R90JT [20] | Infant Face | HRNet | InfAnFace Train [20] |
| FiDIP [8] | Infant Body | DarkPose | SyRIP [8] |

In the adult domain, for facial landmark estimation, the high-resolution network (HRNet) was used. HRNet is an influential multi-resolution convolutional neural network designed to maintain high-resolution representations throughout inference, which is handy for landmark estimation for high-resolution images. For body joint estimation, DarkPose was used, which modifies the standard convolutional heatmap regression approach to landmark estimation with alleviate bias in coordinate representation arising from arbitrary pixel quantization during encoding and decoding. Specifically, a DarkPose model with an HRNet backbone was adopted.

In the infant domain, for facial landmark estimation, the HRNet-R90JT model was used, also built on HRNet and adapted to the infant domain via fine-tuning and data augmentation directed at the unique features of infant faces. Finally, for body joint estimation, a fine-tuned domain-adapted infant pose (FiDIP) model was adopted, which uses synthetic data and domain adversarial methods to adapt body landmark estimation (in this case, the HRNet-backed DarkPose model) to the infant domain.

### Results: Performance Metrics

Often in machine learning, metrics are chosen to enable robust comparison of ever-improving models over time, on large datasets serving as public benchmarks. In this case, because of the relative freshness of the techniques, estimation task, and test set, and because of interest in immediate medical applications, a range of more interpretable metrics were used to provide a sense of performance in an absolute sense, without having to compare with other models. In particular, performance was evaluated using Spearman's ρ correlation coefficient, the binary classification accuracy of predicting whether a quantity is above or below the mean, and the mean absolute error. The root mean squared error is also reported, for purposes of robust future comparison.

Spearman's ρ rank correlation coefficient between two variables is defined as the Pearson's r correlation between the internal rankings for each variable. In the context of deep learning tools trained and tested on imprecise labels and small data domains, high Pearson's r coefficients between predictions and ground truth is too much to ask for, but adopting Spearman's ρ allows relaxation of the requirement for pinpoint accuracy, permitting focus on the relative ranked correctness of the predictions, which is fully compatible with a goal of enabling screening or diagnosis of torticollis. In a similar vein, the binary classification accuracy (BCA) of a set of predictions (\tilde{y}ᵢ)_{i∈1} is assessed relative to the ground truth (yᵢ)_{i∈I} of a given variable, defined simply as the proportion of i in the sample index I for which \tilde{y}ᵢ>μ⇔yᵢ>μ, where μ=μ((\tilde{y}ᵢ)_{i∈I}) is the ground truth sample mean. This coarse measure gauges how often predictions accurately determine whether a quantity is greater or less than the ground truth mean (so unlike Spearman's ρ, it is not purely relative), and its simplicity allows for ease of interpretation. The mean absolute error (MAE) and the root mean squared error (RMSE) are also measured, the former being more interpretable and the latter being more robust for subsequent comparisons.

### Results: Analysis

Table 9 tabulates estimation performance of the geometric measures of symmetry, as gauged by the aforementioned metrics, for both the infant and adult based models. See Table 10 for a guideline for interpreting Spearman's ρ scores.

TABLE 10
INTERPRETATION OF SPEARMAN'S ρ

| Spearman's ρ | Correlation |
|---|---|
| ≥0.70 | Very strong relationship |
| 0.40-0.69 | Strong relationship |
| 0.30-0.39 | Moderate relationship |
| 0.20-0.29 | Weak relationship |
| 0.01-0.19 | No or negligible relationship |

Descriptions apply to both positive and negative values

On the whole, results show that the infant pose estimation models yield predictions of five of the six measures (all except the orbit slopes angle (osa)) with a high degree of fidelity. Spearman's ρ correlations are generally strong or very strong and binary classification accuracies high (75.0-88.9%). Mean absolute errors are within 3° for angles and 0.1 for ratios, and root mean squared errors are low as well. The predictions of the remaining quantity, the orbit slopes angle (osa), only moderately correlate with the ground truth, with ρ=0.36. For the orbit slopes angle, the BCA is no better than random guessing at 50%, but the low MAE and RMSE values suggest the poor classification accuracy may be due to miscalibration. Geometrically, the orbit slopes angle is quite subtle, measuring slight differences between the line connecting the outer cathnuses (eye corners) and the inner cathnuses, and it appears that this distinction is too fine to be reliably measured with the infant landmark estimation model. Turning to the adult pose estimation models, Table 9 shows that for every geometric symmetry measure and every prediction performance metric, the predictions from the adult models fare worse than those from the infant models, with the exception of one tie. Note that this supremacy is not maintained when cross-comparing performances of different measures—for instance, the infant model prediction of face angle (fa) has lower Spearman's ρ and BCA scores than the adult prediction of gaze angle (ga). Indeed, the adult model predictions of half of the measurements—the relative face size (rfs), gaze angle (ga), and habitual head deviation (hhd)—can be considered to be successful under Spearman's ρ and BCA, although their absolute errors (MAE and RMSE) are still high.

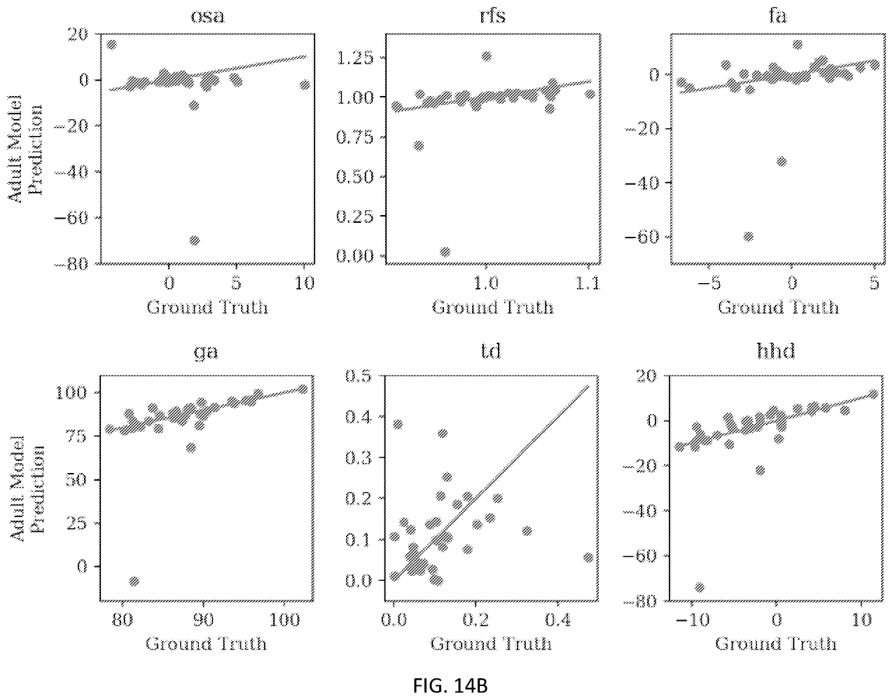

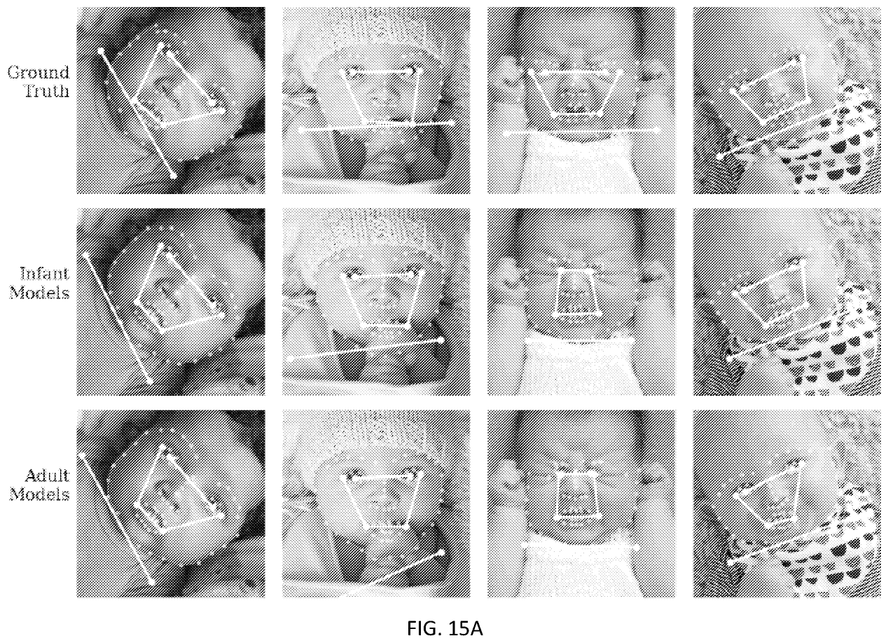

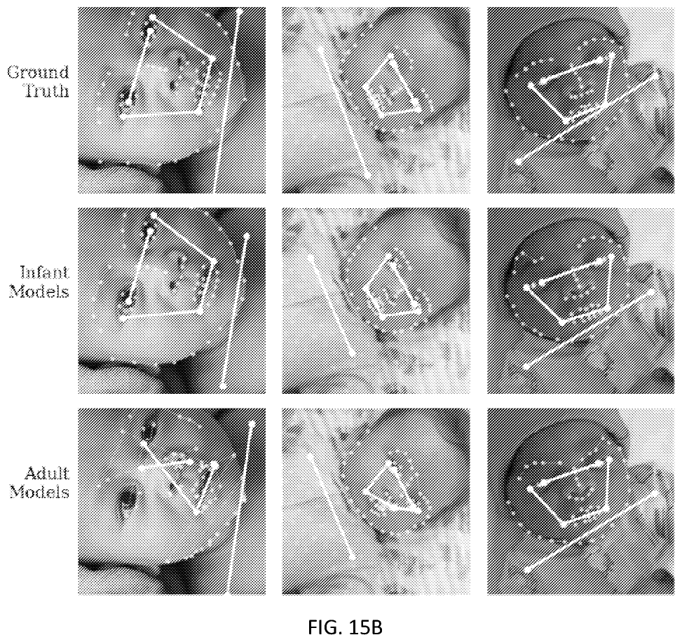

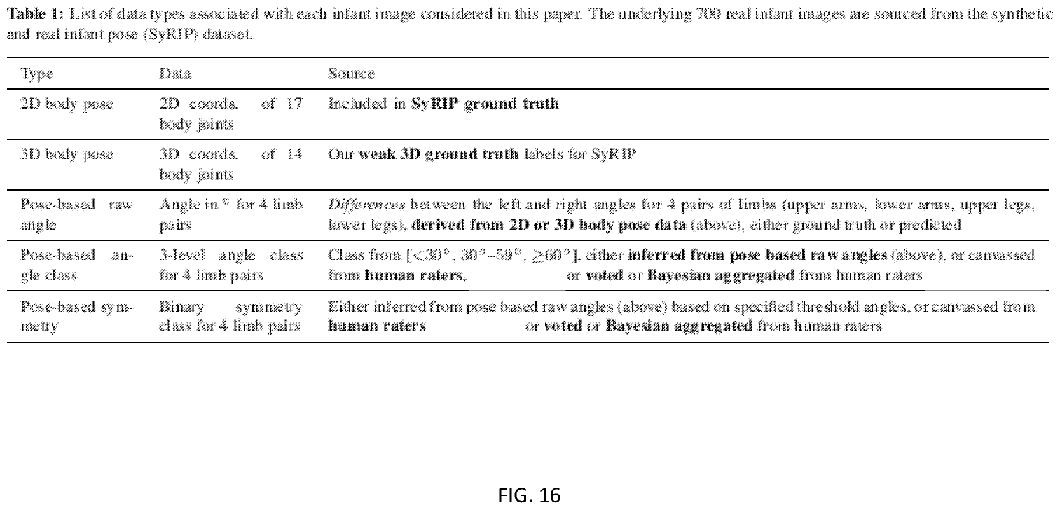

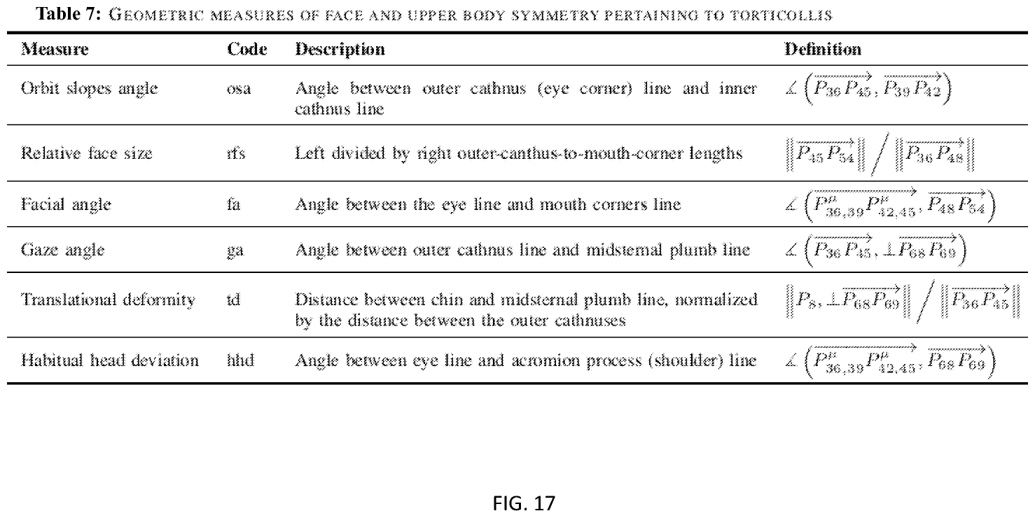

To complement the summary statistics from Table 9, full scatter plots of predicted vs ground truth values for all six measures under both sets of models are included, in FIGS. 14A-14B. The scale of these scatter plots is chosen to highlight the presence of major outlier mispredictions from the adult models, and the subtler performance differences between measurements revealed by Table 9 are harder to perceive.

Finally, a visualization of ground truth and predictions the geometric elements involved in the computation of the six measures of interest can be found FIGS. 15A-15B. The predictions from the infant model appear to be fairly accurate, but since it is hard to visually determine angles and ratios at a glance, it is difficult to make definitive conclusions about performance on this qualitative basis alone. What such figures and the performance scatter plots do make clear, though, is that a major cause of poor performance from the adult pose estimation models stems from a handful of instances of failed facial landmark estimation.

Thus, the inventors have studied the extent to which recent deep learning-based computer vision algorithms designed specifically for the infant domain can measure a set of geometric facial and upper-body symmetry measures previously defined by torticollis researchers, from still images alone. After carefully crafting a test set of infant faces and honing the definitions to line up with standard facial landmarks, the inventors found that most of these measurements can be successfully predicted by pose estimation models designed for infants. The inventors also showed that these models outperform corresponding models designed for and trained on adult data, demonstrating the importance of using tailored neural network models in the data-scarce infant domain.

While example embodiments have been particularly shown and described, it will be understood by those skilled in the art that various changes in form and details may be made therein without departing from the scope of the embodiments encompassed or contemplated herein.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with “consisting essentially of” or “consisting of”.

What is claimed is:

1. A method of screening, diagnosing, or monitoring a disease or condition in a human infant, the method comprising
providing one or more images of the infant;
selecting at least two geometric symmetry measures of the infant indicative of the disease or condition;
analyzing, using both an infant face landmark estimation model and an infant body landmark estimation model of a computer vision system, the one or more images to produce a plurality of landmark values corresponding to the geometric symmetry measures;
determining, by the computer vision system, a geometry of one or more facial structures of the infant and/or a 3D body pose of the infant based on the plurality of landmark values; and
quantifying, based on the geometry of the one or more facial structures of the infant and/or the 3D body pose of the infant, a symmetry of the geometric symmetry measures.

2. The method of claim 1, wherein the disease or condition is torticollis, such as congenital muscular torticollis (CMT), autism spectrum disorder (ASD), or cerebral palsy (CP).

3. The method of claim 1, wherein the step of quantifying comprises determining bilateral postural asymmetry.

4. The method of claim 1, wherein the step of quantifying further comprises using a symmetry classifier to assess, based on body joint angles obtained from the 3D pose determination, one or more symmetry ratings corresponding to the geometric symmetry measures.

5. The method of claim 4, further comprising aggregating, using a Bayesian estimator, a plurality of annotated symmetry ratings corresponding to the one or more symmetry ratings to establish one or more aggregated Bayesian ground truths.

6. The method of claim 5, further comprising comparing each of the symmetry ratings to a corresponding one of the aggregated Bayesian ground truths to calibrate the computer vision system.

7. The method of claim 5, further comprising:
determining, by a maximum-a-posteriori (MAP) estimator, a performance standard applicable to the annotated symmetry ratings; and
evaluating, by applying an expectation maximization algorithm, a performance relative to the performance standard of a rater associated with each of the annotated symmetry ratings.

8. The method of claim 7, wherein the step of aggregating, by the Bayesian estimator, further comprises weighting each of the annotated symmetry ratings according to the evaluated rater performance.

9. The method of claim 7, wherein the evaluated performance is at least partially determined according to an average Cohen's κ agreement between each rater and the other raters.

10. The method of claim 1, wherein the geometric symmetry measures include one or more of orbit slopes angle, relative face size, face angle, gaze angle, translational deformity, habitual head deviation, or combinations thereof.

11. The method of claim 1, wherein the step of analyzing further comprises producing, by the infant face landmark estimation model, produce 68 landmark values.

12. The method of claim 1, wherein the step of analyzing further comprises producing, by the infant body landmark estimation model, a plurality of landmark values corresponding to one or more of an upper arm, a lower arm, an upper leg, a lower leg, or combinations thereof of the infant.

13. The method of claim 12, wherein the plurality of landmark values produced by the infant body landmark estimation model correspond to one or more pairs of limbs of the infant, the one or more pairs of limbs of the infant including one or more of an upper right arm and upper left arm pair, a lower right arm and lower left arm pair, an upper right leg and upper left leg pair, a lower right leg and lower left leg pair, or combinations thereof.

14. The method of claim 13, wherein the step of quantifying a symmetry of the of the geometric symmetry measures further comprises determining an angle difference across each of the one or more pairs of limbs.

15. The method of claim 14, further comprising comparing the angle difference for each of the one or more pairs of limbs to a corresponding threshold angle.

16. The method of claim 15, further comprising classifying each of the pairs of limbs as symmetrical or asymmetrical, wherein;
a symmetrical classification indicates that a corresponding one of the pairs of limbs has a determined angle difference within the corresponding angle threshold; and
an asymmetrical classification indicates that the corresponding one of the pairs of limbs has a determined angle difference exceeding the corresponding angle threshold.

17. The method of claim 14, further comprising assigning each of the pairs of limbs to an angle class.

18. The method of claim 17, wherein the angle classes include one or more of <30°, ≥30°, <60°, ≥60°, 30°-59°, or combinations thereof.
